(19) United States
(12) Patent Application Publication
(10) Pub. No.: US 2022/0364120 A1
(43) Pub. Date: Nov. 17, 2022

(54) DROPLET MICROFLUIDIC PLATFORM FOR THE ENHANCED DNA TRANSFER BETWEEN MICROBIAL SPECIES

(71) Applicants: The Texas A&M University System, College Station, TX (US); U.S. Army Research Laboratory, Adelphi, MD (US)

(72) Inventors: Arum Han, College Station, TX (US); Jose Wippold, Bryan, TX (US); Bryn L. Adams, Silver Spring, MD (US)

(21) Appl. No.: 17/745,488

(22) Filed: May 16, 2022

Related U.S. Application Data
(60) Provisional application No. 63/189,674, filed on May 17, 2021.

(51) Int. Cl. C12N 15/88; B01L 3/00

(52) U.S. Cl. CPC C12N 15/88; B01L 3/502784; B01L 2200/16; B01L 2400/084; B01L 2200/0673; B01L 2300/0636

(57) ABSTRACT

In an embodiment, the present disclosure pertains to a microfluidic platform composed of a droplet generator having an entry point for donor particles and target particles, a first droplet incubation chamber in fluid communication with the droplet generator, a droplet detection functionality to allow for analysis of the inner content of droplets, and a droplet sorting functionality to allow for the separation of droplets based on the analysis of the inner content of droplets. In another embodiment, the present disclosure pertains to a method for cell-to-cell DNA, RNA, or other genetic material transfer through use of a water-in-oil emulsion microdroplet-based microfluidic platform for automation and high throughput identification or screening of genetic transfer outcomes utilizing the microfluidic platforms as disclosed herein.

### CROSS-REFERENCE TO RELATED APPLICATIONS

This patent application claims priority from, and incorporates by reference the entire disclosure of, U.S. Provisional Application No. 63/189,674 filed on May 17, 2021.

### STATEMENT REGARDING FEDERALLY SPONSORED RESEARCH

This invention was made with government support under W911NF-17-2-0144 awarded by the Army Research Laboratory. The government has certain rights in the invention.

### TECHNICAL FIELD

The present disclosure relates generally to DNA transfer platforms and more particularly, but not by way of limitation, to two-phase microfluidic devices to enhance and screen for efficient DNA transfer between cells.

### BACKGROUND

This section provides background information to facilitate a better understanding of the various aspects of the disclosure. It should be understood that the statements in this section of this document are to be read in this light, and not as admissions of prior art.

A significant hurdle for the widespread implementation of synthetic biology (synbio) is the challenge of high throughput DNA introduction into cells which continues to be a barrier for the integration of novel chassis species in a variety of biotechnology fields. Common approaches to DNA uptake, such as competent cell transformation and electroporation, are typically only amenable to a very small subset of microbial species and leave the vast majority of microbial life genetically inaccessible.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a microfluidic platform composed of a droplet generator having an entry point for donor particles and target particles, a first droplet incubation chamber in fluid communication with the droplet generator, a droplet detection functionality to allow for analysis of the inner content of droplets, and a droplet sorting functionality to allow for the separation of droplets based on the analysis of the inner content of droplets. In some embodiments, the first droplet incubation chamber is configured to allow interaction between the donor particles and the target particles. In some embodiments, the droplet detection functionality is at least one of paired with or incorporated on the microfluidic platform. In some embodiments, the droplet sorting functionality is at least one of paired with or incorporated on the microfluidic platform.

In some embodiments, the droplet generator has a first droplet generator for the donor particles and a second droplet generator for the target particles in fluid communication with the first droplet incubation chamber thereby allowing flow of droplets containing the donor particles and droplets containing the target particles into the first droplet incubation chamber.

In some embodiments, the droplet generator has a first droplet generator for the donor particles and a second droplet generator for the target particles in fluid communication with the first droplet incubation chamber. In some embodiments, the first droplet incubation chamber includes separate donor and target particle incubation chambers thereby allowing flow of droplets containing the donor particles into the donor incubation chamber and droplets containing the target particles into the target droplet incubation chamber.

In some embodiments, the microfluidic device further includes an induction media or signal-activating media droplet generator having an entry point for at least one of an induction media or a supplemental media, a first droplet merging region in fluid communication with the first droplet incubation chamber, and a second droplet incubation chamber in fluid communication with the first droplet merging region. In some embodiments, the induction or signal-activating media droplet generator is configured to release at least one of the induction media or the supplemental media at a point between the first droplet incubator and the first droplet merging region. In some embodiments, the second droplet incubation chamber is configured to allow for signal induction.

In some embodiments, the microfluidic platform further includes a signal amplification media droplet generator having an entry point for signal amplification media, a second droplet merging region in fluid communication with the second droplet incubation chamber, and a third droplet incubation chamber in fluid communication with the second droplet merging region. In some embodiments, the signal amplification media droplet generator is configured to release the signal amplification media at a point between the second droplet incubation chamber and the second droplet merging region. In some embodiments, the third droplet incubation chamber is configured to allow for signal amplification.

In some embodiments, the microfluidic platform further includes a first outlet and a second outlet. In some embodiments, the first outlet is configured to release waste particles displaying no transfer. In some embodiments, the second outlet is configured to recover particles displaying successful transfer. In some embodiments, the donor particles and target particles are at least one of DNA, RNA, cells, small molecules, or combinations thereof. In some embodiments, the donor particles and target particles are cells having DNA. In some embodiments, the microfluidic platform further includes a first outlet and a second outlet. In some embodiments, the first outlet is configured to release waste cells displaying no DNA transfer (no genetic material transfer). In some embodiments, the second outlet is configured to recover cells displaying successful DNA transfer (a genetic material transfer).

In some embodiments, the microfluidic platform further includes microfluidic channels connecting each chamber of the microfluidic platform. In some embodiments, the microfluidic channels are sloped. In some embodiments, at least one of an X-, Y-, or Z-direction side of the microfluidic channels have a slope that can include, without limitation, up, down, and combinations thereof. In some embodiments, at least one of an X-, Y-, or Z-direction side of the microfluidic channels slope has a spatial combination. In some embodiments, at least one of the microfluidic channels become at least one of shallower, deeper, wider, narrower, or combinations thereof in relation to a first point of the microfluidic channel and a second point of the microfluidic channel.

In an additional embodiment, the present disclosure pertains to a microfluidic platform including a droplet generator having an entry point for donor particles and target particles, a first droplet incubation chamber in fluid communication with the droplet generator, an induction media droplet generator having an entry point for induction media, a first droplet merging region in fluid communication with the first droplet incubation chamber, a second droplet incubation chamber in fluid communication with the first droplet merging region, a signal amplification media droplet generator having an entry point for signal amplification media, a second droplet merging region in fluid communication with the second droplet incubation chamber, a third droplet incubation chamber in fluid communication with the second droplet merging region, a first outlet, and a second outlet. In some embodiments, the first droplet incubation chamber is configured to allow interaction between the donor particles and the target particles. In some embodiments, the induction media droplet generator is configured to release the induction media at a point between the first droplet incubator and the first droplet merging region. In some embodiments, the second droplet incubation chamber is configured to allow for signal induction. In some embodiments, the signal amplification media droplet generator is configured to release the signal amplification media at a point between the second droplet incubation chamber and the second droplet merging region. In some embodiments, the third droplet incubation chamber is configured to allow for signal amplification. In some embodiments, the first outlet is configured to release waste particles displaying no transfer. In some embodiments, the second outlet is configured to recover particles displaying successful transfer.

In some embodiments, the donor particles and target particles are at least one of DNA, RNA, cells, small molecules, or combinations thereof. In some embodiments, the donor particles and target particles are cells having DNA. In some embodiments, the first outlet is configured to release waste cells displaying no DNA transfer. In some embodiments, the second outlet is configured to recover cells displaying successful DNA transfer.

In some embodiments, each droplet generator, each droplet merging region, and each droplet incubation chambers are fluidly connected via microfluidic channels. In some embodiments, the microfluidic channels have a portion including at least one of a sloped region, a flat region, or combinations thereof. In some embodiments, at least one of an X-, Y-, or Z-direction side of the microfluidic channels slope in a spatial combination. In some embodiments, at least one of the microfluidic channels become at least one of shallower, deeper, wider, narrower, or combinations thereof in relation to a first point of the microfluidic channel and a second point of the microfluidic channel.

In a further embodiment, the present disclosure pertains to a method for cell-to-cell DNA, RNA, or other genetic material transfer through use of a water-in-oil emulsion microdroplet-based microfluidic platform for automation and high throughput identification or screening of genetic transfer outcomes. In general, the method includes adding donor cells and target cells to a microfluidic platform thereby forming droplets, adding an induction media to the microfluidic platform, adding a signal amplification media to the microfluidic platform, detecting functionality to allow for analysis of inner content of the droplets, and sorting the droplets to allow for separation of the droplets based on the analysis of inner content of the droplets.

In some embodiments, the microfluidic device is composed of a droplet generator having an entry point for the donor cells and the target cells, a first droplet incubation chamber in fluid communication with the droplet generator, an induction media droplet generator having an entry point for the induction media, a first droplet merging region in fluid communication with the first droplet incubation chamber, a second droplet incubation chamber in fluid communication with the first droplet merging region, a signal amplification media droplet generator having an entry point for the signal amplification media, a second droplet merging region in fluid communication with the second droplet incubation chamber, a third droplet incubation chamber in fluid communication with the second droplet merging region, a first outlet, and a second outlet. In some embodiments, the first droplet incubation chamber is configured to allow interaction between the donor cells and the target cells. In some embodiments, the induction media droplet generator is configured to release the induction media at a point between the first droplet incubator and the first droplet merging region. In some embodiments, the second droplet incubation chamber is configured to allow for signal induction. In some embodiments, the signal amplification media droplet generator is configured to release the signal amplification media at a point between the second droplet incubation chamber and the second droplet merging region. In some embodiments, the third droplet incubation chamber is configured to allow for signal amplification. In some embodiments, the first outlet is configured to release waste cells displaying no DNA transfer. In some embodiments, the second outlet is configured to recover cells displaying successful DNA transfer.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A illustrates a diagram of an example device housing all the microdroplet techniques on a single platform according to aspects of the present disclosure.

FIG. 1B illustrates an example embodiment in which there exist separate droplet generators for the donor and target cells flowing into a single chamber according to aspects of the present disclosure.

FIG. 1C illustrates an example embodiment in which there exist separate droplet generators for the donor and target cells flowing into separate chambers according to aspects of the present disclosure.

FIG. 2 illustrates simplified XPORT (a genetically engineered cell) DNA conjugation processing.

FIG. 3 illustrates a simplified example fabrication process according to an aspect of the present disclosure.

FIG. 4 illustrates an example diagram demonstrating how dielectrophoretic (DEP) droplet sorting is conducted following fluorescence detection of the droplet content according to an aspect of the present disclosure.

FIG. 5 illustrates an example diagram of one embodiment of how to achieve the pairing of a microfluidic gradient generator with a microfluidic droplet generator such that donor cell to target cell ratios inside the generated droplet span an intended range within a single experimental trial.

FIGS. 6A-6B illustrate an overview schematic comparing a conventional XPORT DNA transfer approach (FIG. 6A) and an example configuration of a DNA ENhanced TRAnsfer Platform (DNA ENTRAP) microfluidic approach (FIG. 6B).

FIG. 7 illustrates a potential alternative workflow of the DNA ENTRAP microfluidic system according to an aspect of the present disclosure.

FIG. 8 illustrates a schematic comparing a traditional benchtop approach (bottom) when compared to an example workflow of a DNA ENTRAP system (top) to demonstrate the compatibility of a DNA ENTRAP system with liquid handling systems, enabling a fully automated protocol.

FIG. 9 illustrates experimental results comparing XPORT donor cell viability over the course of 12 hours and receiver cell viability over the course of 12 hours in droplet format. Standard deviation denoted by error bars obtained from triplicate experiments.

FIGS. 10A-10B illustrate data demonstrating the superiority of the microfluidic systems of the present disclosure when compared to conventional benchtop methods. FIG. 10A shows results from a 3:1 ratiometric donor to recipient in-droplet pairing. FIG. 10B shows results from a 1:1 ratiometric donor to recipient in-droplet pairing.

FIG. 11A illustrates an example workflow schematic for the confirmation assays according to an aspect of the present disclosure.

FIG. 11B illustrates a depiction of an example DNA payload transferred from a donor cell to a receiver cell according to an aspect of the present disclosure.

FIG. 12 illustrates quantification of absorbance readings relating to melanin production from cells that underwent a DNA ENTRAP workflow in which the capability to produce melanin (via a tyrosinase gene that facilitates the conversion of tyrosine to 3,4-dihydroxyphenylalanine (DOPA) and to dopaquinone followed by a subsequent oxidation step) delivered from donor cell to receiver cell enables subsequent melanin production. Dilutions plotted along the X-axis indicate the addition of phosphate buffered saline (PBS) to the original experimental samples and were done to eliminate the potential of absorbance saturation due to excessively high melanin presence.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

A synthetic biology (synbio) tool, XPORT (a genetically engineered cell), was leveraged and adapted for use in a novel high throughput, microfluidic platform to address the challenge of high throughput DNA introduction into cells which continues to be a barrier for the integration of novel chassis species in a variety of biotechnology fields. For example, standard laboratory practices for performing methods of gene transfer in microbes can be time consuming, and the best transfer conditions may vary from organism to organism.

A new system named DNA ENhanced TRAnsfer Platform (DNA ENTRAP), a water-in-oil emulsion droplet-based microfluidic platform that generates nano-bioreactors is described herein. Many components of the platforms as disclosed herein are highly automatable to further streamline experimental processing. These systems and methods provide the capability to screen multi-parameter conditions that streamline XPORT-mediated genetic transfer at unprecedented rates by miniaturizing and automating many elements of the XPORT process. Furthermore, the DNA ENTRAP technology can be utilized to screen large library of cells (either donor cells or receiver cells) for various purposes, including identifying receiver cells that can efficiently receive DNA. Device configurations allow for modular control of the donor:recipient ratios within the microdroplets by pairing an enabling device geometry along with custom Laboratory Virtual Instrument Engineering Workbench (LabVIEW) automation, or computer-programmed automation tools. The design and fabrication of DNA ENTRAP is described herein, and an enhanced XPORT-mediated genetic transfer between two Bacillus subtilis strains is demonstrated. Furthermore, increased DNA transfer efficiency over current, benchtop XPORT processes, as well as increased output using the typical automated facets inherent to microfluidic system control is demonstrated.

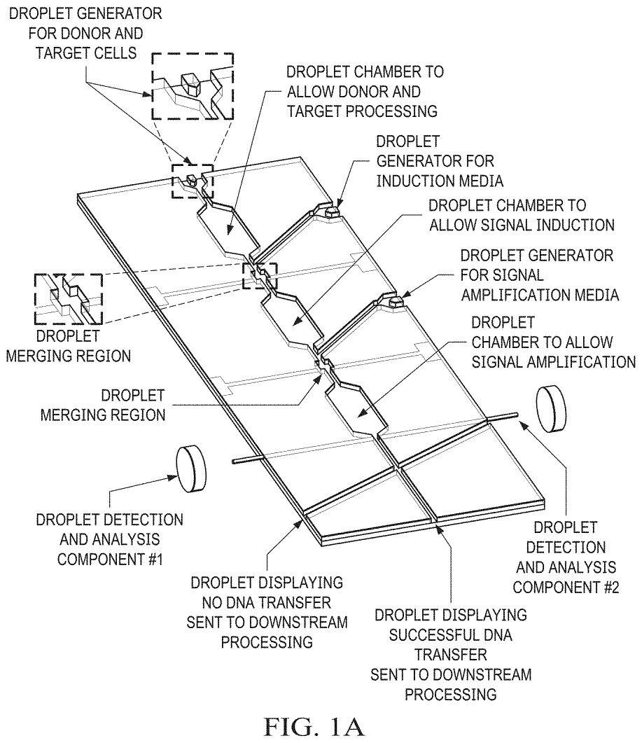

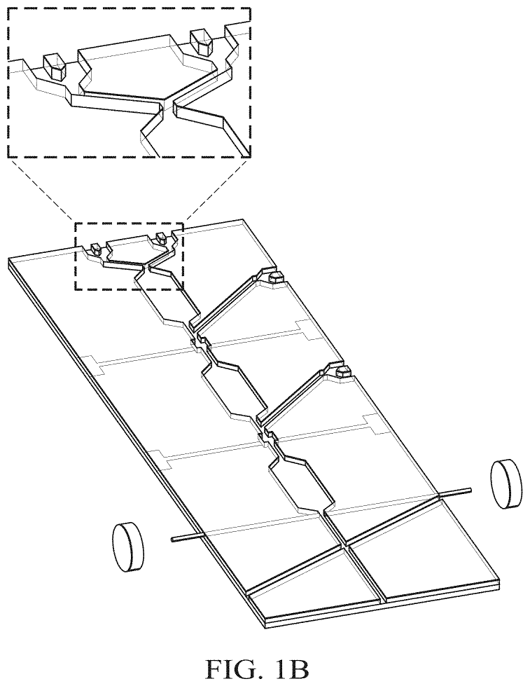

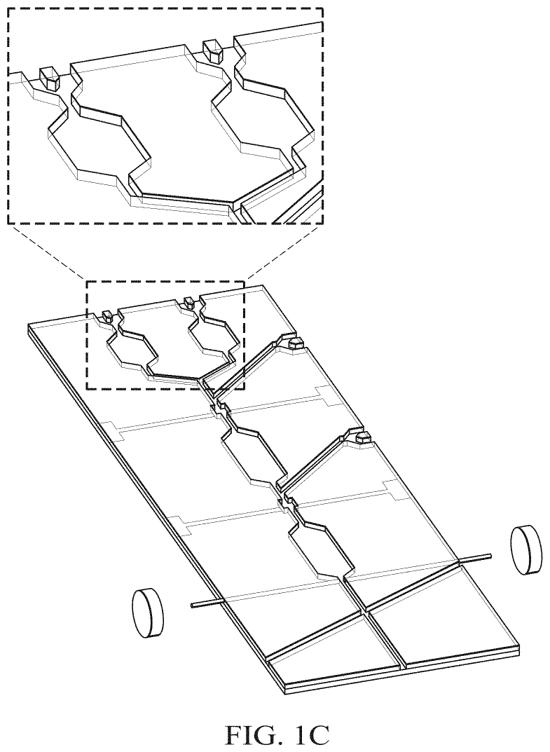

FIG. 1A shows a diagram of an example device housing all the microdroplet techniques on a single platform. It should be noted that this, and the following schematics, are just some possible device configurations according to various aspects of the disclosure. FIG. 1B shows an example embodiment in which there exist separate droplet generators for the donor and target cells flowing into a single chamber. FIG. 1C shows a third example embodiment in which there exist separate droplet generators for the donor and target cells flowing into separate chambers. Such embodiments are described in further detail below. Additionally, in some embodiments, the device may have any combination of droplet manipulation functionalities incorporated into the platform with others not specifically stated herein, but readily envisioned by one of ordinary skill in the art. In some embodiments, separate droplet generators and separate incubation chambers exist for both the donor cells/particles and the receiver cells/particles. As used herein, the term particle can refer to DNA, RNA, proteins, cells, cells having DNA, cells having RNA, small molecules, chemicals, particles (such as cells) that have any genetic material, and combinations of the same and like. In another embodiment, separate droplet generators for both donor cells and receiver cells exist and are in fluid communication with a single incubation chamber for both populations of droplets. In such embodiments, a first droplet merging region in fluid communication with the separate or common incubation chambers is used to create a single droplet with both donor cells and receiver cells. In yet another embodiment, a common droplet generator forming donor cell and receiver cell droplets exists and is in fluid communication with a droplet incubation chamber. Fluidically connected to this, a droplet generator forming induction media rich droplet exists and forms fluidic connection with a microfluidic channel combining the populations of donor and receiver-rich droplets with the induction media-rich droplets. In other embodiments, the newly formed droplet is in direct fluid communication with another channel in which a droplet generator producing an induction media-rich droplet that feed into a droplet merging region to combine the donor-and-receiver droplet with the newly formed induction media-rich droplet.

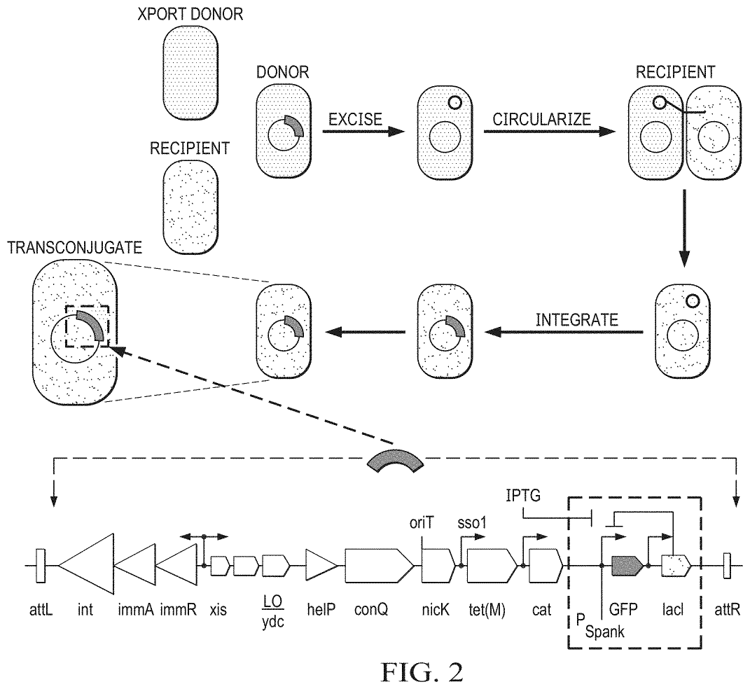

The capability to transfer DNA into another organism, whether natural or synthetic, is a fundamental technology that is needed and can benefit in the broad area of synthetic biology, biotechnology, microbiology, medicine, and life science in general. Significant advances have been made in organism engineering using a variety of synbio tools and systems. However, the introduction of DNA into novel chassis organisms, particularly those that are uncharacterized, remains a major obstacle in advancing the state of the art in the field. There are several common and efficient methods to introduce DNA into microbes, including chemical competence, protoplasting, electroporation, and E. coli mediated conjugation. However, these methods only work for a handful of domesticated chassis microbes, thus limiting the range of microbes that may serve as synbio host cells. To address this barrier, a broad host conjugation-based DNA transfer tool, XPORT, was previously developed and published (FIG. 2). In this approach, the conjugation machinery, including the integrative and conjugative element (ICE) endogenous to Bacillus subtilis JH642, was engineered to controllably deliver genetic circuits to non-traditional and undomesticated chassis microbes. XPORT-mediated conjugation requires that the DNA donor and recipient cells be in direct physical contact to facilitate DNA transfer, and often the ratio of donor to recipient cells must be quite high (e.g., 10⁴:1) to ensure success. While XPORT introduces a promising method for engineering undomesticated bacteria, there are limitations in that this technique is performed exclusively by hand (i.e., no robotics or automations are involved) and is also very time and labor intensive, severely restricting the number of conjugations that can be attempted at one time. Thus, this system is ideal for adaptation into a high-throughput, automated microfluidic system, as the nature of XPORT-mediated DNA transfer lends itself well to miniaturization into microdroplets. The need for donor and recipient cells to be in close proximity and the ability to automate a multi-step process is expected to increase DNA transfer efficiency and scale up XPORT for use as a rapid discovery method.

Droplet microfluidic technology has demonstrated utility as a screening mechanism for numerous biological applications using various types of bacteria encapsulated in droplets. This technology has been used to characterize bacterial growth byproducts, degrees of bacterial pathogenicity, environmental bacterial growth rates, and mechanisms of bacterial antibiotic resistance, to name a few. Droplet microfluidics has widely been used in a range of cell and molecule screening applications, and is therefore highly compatible for realizing the goal of a platform that enhances DNA transfer via conjugation-based approaches. Microdroplet technology offers several advantageous characteristics, such as low volume enclosure of encapsulated cells, a high quantity of droplet nanoreactors produced in short periods of time, and ability to incorporate complex liquid manipulation protocols into large parallel droplet experimentation that aides in streamlining complex synbio protocols. In recent years, droplet microfluidic technology has begun to be used more routinely in bioengineering and synthetic biology applications, but its use to facilitate DNA transfer and genetic engineering remains limited. Currently, there has been only one demonstration of in-droplet DNA transfer between bacterial strains. This demonstration describes the encapsulation of Streptococcus pneumonia strains capable of DNA transformation via pneumococcal cell-cell lytic attacks using three different antibiotic resistance genes. The demonstration showed successful in-droplet transformation, followed by recovery of the recombinants from the droplets through selective plating. The use of droplet microfluidic technology in this demonstration was successful because it brings donor and recipient organisms into close proximity via an enclosed droplet. However, a high (5%) surfactant concentration was used, leading to lower than expected cellular viability in droplets. The relatively small droplet, despite the claimed functionality for optimizing pneumococcal cell-cell lytic attacks, inherently decreased the ratio of inner content media volume to surrounding surfactant, thus resulting in less optimal conditions for cell viability. From a device standpoint, the system design did not enable an on-chip, in-droplet viability assessment. In brief, no droplet microfluidic platform has demonstrated, characterized, or optimized a bacterial conjugation system within microdroplets. The present disclosure aims to resolve a major bottleneck limiting the development of novel synbio chassis with a throughput and testing capability sufficient to address the fundamental limitations of time and manual work power. It also enables downstream automation through the development of LabVIEW controlled fluid flows along with the incorporation of various liquid handling steps, such as droplet merging and automated analysis through droplet content detection and droplet sorting.

To this end, the DNA ENTRAP systems and methods presented herein simplify, enhance, and increase the throughput of XPORT-mediated DNA transfer in B. subtilis, as an example, using a droplet microfluidics approach. No system currently exist that enables high-throughput, multi-parametric experimentation of XPORT-mediated gene transfer. XPORT is a specialized Bacillus subtilis strain engineered to enable inducible control over conjugation events that integrate delivered DNA into the chromosome of a recipient cell, including unidentified environmental samples. The DNA ENTRAP microfluidic systems presented herein have demonstrated efficient XPORT-mediated nucleic acid transfer and thus establishes itself as the first high-throughput microfluidic system capable of screening multi-parametric conditions needed to streamline the development of genetic transfer protocols. The droplet microfluidic system enhances cell-cell interaction while also increasing the probability of multiple cell-contact events through the co-encapsulation of competent XPORT donor cells with activated conjugative machinery in nanoscale bioreactors with the recipient cells. The platforms disclosed herein achieved a 3:1, donor cells to recipient cell conjugation efficiencies improvement of greater than 200% when compared to a traditional benchtop method at 1 hr co-incubation time-point. Furthermore, at every tested co-incubation period and donor to host ratios, the DNA ENTRAP systems and methods outperformed that of traditional benchtop methods. Taken together, these results establish the DNA ENTRAP microfluidic systems and methods as powerful engines for developing horizontal gene transfer systems and protocols between donor cells and recipient cells that rapidly screen, not only donor and recipient cell compatibility, but also cell-to-cell ratios and co-incubation time for optimum results. It is envisioned that the DNA ENTRAP systems will readily become an important tool in the domestication of natural and artificial organisms in order to aid the bioengineering of desired traits and adaptations into entities in which the incorporation of these characteristics may not otherwise be possible.

The DNA ENTRAP systems and methods presented herein can enable rapid identification of the best XPORT-mediated DNA transfer conditions from a variety of different parameters, such as growth parameters, strain growth state, and other pre-determined physiological conditions that impact the ability of conducting conjugative DNA transfer. These platforms can be utilized to screen environmental microbes that can accept genetic materials from the donor cells. In another example, the platform can be utilized for screening and analyzing highly complex cellular libraries. Even with a modest system throughput of tens of droplets per second, the DNA ENTRAP system can screen a population size of 10⁶ environmental isolates in under 4 days. Using the conventional agar-plate based strategy and approach, with a day and night shift team of 25 scientists working 12 hour shifts each (50 plates/day), this same 10⁶ environmental isolate population is expected to be screened in 54.8 years.

As such, the present disclosure relates to a system to enhance cell-to-cell DNA transfer through the use of a water-in-oil emulsion microdroplet-based microfluidic platform for automatic and high throughput identification and/or screening of DNA transfer conditions and outcomes. This directly addresses the need for high-throughput, reliable, accurate, and automatic systems that can be employed to streamline the labor intensive and tedious genetic modification processes into a rapid discovery engine. The end result is a unique system that overcomes the time consuming, labor intensive, and inaccurate process currently being used, and simultaneously enables multi-parameter adjustments and screening of cell-to-cell DNA transfer conditions and their outcome.

The systems of the present disclosure feature a two-phase emulsion microfluidic system (i.e. droplet microfluidic) that compartmentalizes the cellular components into a confined space, which serves as the cell-to-cell DNA transfer micro-bioreactor. For example, by putting two cells, the donor cell and receiver cells, inside a single microdroplet, the DNA transfer efficiency can be enhanced due to their close proximity. More importantly, the number of each cell type (donor and receiver cells) within the droplet, co-cultivation conditions, and size of the droplet (thus the average distance between the cells) can all be controlled with high accuracy, enabling the rapid identification of the conditions and/or cell strains that show the best transfer efficiencies.

As shown in FIGS. 1A-1C, the DNA ENTRAP droplet microfluidic system can be composed of series of separate but interconnected droplet microfluidic functions to accomplish rapid and direct DNA transfer as well as characterization/identification of the optimum assay conditions. First, cell-encapsulated microdroplet can be formed (referred to as droplet generation), where donor-cell-encapsulated microdroplets and receiver-cell-encapsulated microdroplets are generated (creating two populations of microdroplets). Methods of droplet generation can be active (involving outside energy or stimulus) or passive (operating without the need of outside energy), and can occur via microfluidic cross-flow designs or T-, K-, or V-junction designs commonly used in the field. Droplets can be generated on-chip to a wide variety of sizes, ranging anywhere from single micron diameters to several hundred-micron diameters.

Second, depending on the need, these microdroplets can be incubated (referred to as droplet incubation) to expand the number of cells in each droplet, and/or adjust the growth state of the cells in each droplet (e.g., lag phase, exponential growth phase). This microdroplet cultivation can occur either on an on-chip cultivation chamber or an off-chip cultivation chamber.

Third, microdroplets from each population (donor cell population and receiver cell population) can be selectively combined one by one (referred to as droplet merging), creating a single microdroplet that contains both donor and receiver cells. Here, the ratio of donor and receiver cells, as well as their numbers, can be controlled by various parameters, for example the number of cells initially encapsulated, duration of cultivation to expand the initial cells encapsulated within the droplets, and the like. Further control can be achieved by merging more than one receiver- or donor-cell droplets (not only 1:1, but for example, 2:1, 1:2, 5:1, 1:5, etc.), either through a single droplet merging step or through a series of repeated droplet merging steps. This droplet merging step can be achieved using any combination of active or passive droplet merging techniques commonly used in the field.

Fourth, the inner contents of the microdroplet can be analyzed (referred to as droplet detection) to characterize the DNA transfer efficiency. Droplet detection can be achieved by incorporating a sensing region into the device or by having the sensing region in close proximity to the microfluidic channel. Sensing can be achieved either optically through brightfield or luminescence and/or fluorescence detection and/or imaging, or electrically through impedance, conductance, or resistance measurements. Optical methods can include sensors to detect light emitted by the inner contents of the microdroplets ranging a span of different wavelengths or a single wavelength. Detectors can include charged coupled devices (CCDs), photodiodes (PD), photomultiplier tubes (PMT), and/or single photon counters (SPC).

Finally, based on the readout from the sensor, a target droplet that is deemed to be of high interest can be sorted (referred to as droplet sorting) and collected on-chip or off-chip for further analysis. Droplet sorting can be achieved using either passive or active methods. Passive methods can include, for example, differential size sorting, bifurcation-induced droplet sorting, and combinations of the same and like. Active droplet sorting methods can include, for example, dielectrophoretic (DEP) force-based sorting (discussed further below), acoustic force-based sorting, pneumatic force-based sorting, magnetic force-based sorting, thermal-based droplet sorting, and combinations of the same and like. Many of the unit functions of the droplet manipulation techniques have been characterized and established, or are well known and accepted in the field of microfluidics and lab-on-a-chip technology.

As such, in an embodiment, the present disclosure pertains to a microfluidic platform composed of a droplet generator having an entry point for donor particles and target particles, a first droplet incubation chamber in fluid communication with the droplet generator, a droplet detection functionality to allow for analysis of the inner content of droplets, and a droplet sorting functionality to allow for the separation of droplets based on the analysis of the inner content of droplets. In some embodiments, the first droplet incubation chamber is configured to allow interaction between the donor particles and the target particles. In some embodiments, the droplet detection functionality is at least one of paired with or incorporated on the microfluidic platform.

In some embodiments, droplet detection and droplet sorting functionalities are serially connected through fluidic communication. In some embodiments, these functionalities might be housed on a separate chip connected via tubing or some other communication mechanism.

In some embodiments, the droplet sorting functionality is at least one of paired with or incorporated on the microfluidic platform. In some embodiments, the droplet generator has a first droplet generator for the donor particles and a second droplet generator for the target particles in fluid communication with the first droplet incubation chamber thereby allowing flow of the donor particles and the target particles into the first droplet incubation chamber. In some embodiments, the droplet generator has a first droplet generator for the donor particles and a second droplet generator for the target particles in fluid communication with the first droplet incubation chamber. In some embodiments, the first droplet incubation chamber includes separate donor and target particle incubation chambers thereby allowing flow of the donor particles into the donor incubation chamber and the target particles into the target droplet incubation chamber. In some embodiments, the microfluidic platform further includes a first outlet and a second outlet. In some embodiments, the first outlet is configured to release particles displaying no transfer to waste. In some embodiments, the second outlet is configured to recover particles displaying successful DNA transfer. In some embodiments, the donor particles and target particles are at least one of DNA, RNA, cells, small molecules, or combinations thereof. In some embodiments, the donor particles and target particles are cells having DNA. In some embodiments, the microfluidic platform further includes a first outlet and a second outlet. In some embodiments, the first outlet is configured to release waste cells displaying no DNA transfer as waste. In some embodiments, the second outlet is configured to recover cells displaying successful DNA transfer.

In addition to the steps described above, depending on the particular assay and/or cells used, additional reagents, including induction media and/or signal amplification media, can be generated into a droplet format and merged with the co-culture droplets (FIGS. 1A-1C). Alternatively, these reagents may be directly inserted into the droplet using a technique called “pico-injection”.

Therefore, in some embodiments, the microfluidic device further includes an induction media or signal-activating media droplet generator having an entry point for at least one of an induction media or a supplemental media, a first droplet merging region in fluid communication with the first droplet incubation chamber, and a second droplet incubation chamber in fluid communication with the first droplet merging region. In some embodiments, the induction or signal-activating media droplet generator is configured to release at least one of the induction media or the supplemental media at a point between the first droplet incubator and the first droplet merging region. In some embodiments, the second droplet incubation chamber is configured to allow for signal induction. In some embodiments, the microfluidic platform further includes a signal amplification media droplet generator having an entry point for signal amplification media, a second droplet merging region in fluid communication with the second droplet incubation chamber, and a third droplet incubation chamber in fluid communication with the second droplet merging region. In some embodiments, the signal amplification media droplet generator is configured to release the signal amplification media at a point between the second droplet incubation chamber and the second droplet merging region. In some embodiments, the third droplet incubation chamber is configured to allow for signal amplification.

As discussed above, various other configurations are readily envisioned. For example, in some embodiments, a droplet merging region can be utilized that combines both a donor and target cell droplet, allow for incubation, and then proceed downstream to meet and merge with a signal induction droplet. In such instances, a droplet incubation chamber can be included prior to the droplet merging region that combines the donor and target cell droplets. As such, in some embodiments, a droplet merging region exists in fluid communication with the donor and targets particles droplet generators prior to communicating fluidically with the downstream signal induction droplets. In some embodiments, a droplet incubation chamber for the donor and target cells exists prior to a droplet merging region for the donor and target cell droplets. In some embodiments, there exists a droplet incubation chamber immediately following the donor and target cell droplets and prior to the signal induction droplet generator. Additionally, in some embodiments, the devices of the present disclosure can include a first droplet merging region in fluid communication with the droplet incubation chambers containing separate populations of donor cell droplets and receiver cell droplet droplets. In some embodiments, the devices of the present disclosure can include a first droplet merging region in fluid communication with the single droplet incubation chamber containing a mixed population of donor cell droplets and receiver cell droplets.

To more easily adjust the DNA-transfer conditions to be tested in an automated way, instead of generating the donor and receiver cell droplets separately, donor cells and receiver cells can be co-flown in liquid medium and the flow merged into a single stream, and then cell-encapsulated droplets be generated. Here, the number of donor and receiver cells and their ratios can be controlled by the starting concentration of each cell type, flow rate between the two flow streams, and combinations of the same and like. The controlled ratio can be achieved with what has been termed a “gradient generation”. In this scenario, the donor cell flowrate and the receiver cell flowrate are independently controlled and then the two flows merged into a single flow, which can be the input flow into a droplet generation junction. When these flow rates are identical, a 1:1 concentration ratio between, for example, A cells and B cells, each input organism type is expected, assuming that their concentrations are identical. As flowrate A decreases with respect to flowrate B, more B organisms are expected to be encapsulated in a droplet than A organisms. In contrast, as flowrate A increases with respect to flowrate B, more A organisms are expected to be encapsulated in a droplet than B organisms. By gradually changing the flowrate ratios, a series of droplets in which the A:B cell ratio changes can be generated, allowing testing a large combinations of cell ratios in one experimental run.

The microfluidic system having either sloped or constant-height microchannels can be made of various polymer materials such as polydimethylsiloxane (PDMS), thermoplastic, polymethyl methacrylate (PMMA), polycarbonate (PC), or even glass. The microfabrication process enables the creation of a microfluidic channel where the height (or depth) maintains a constant value in the positive or negative Z-directions, or a curved and/or sloped microfluidic channel, or a microfluidic channel where the height (or depth) can be changed in multiple (two or more) steps. In other words, this is a microfluidic channel where the X-, Y-, and Z-direction side of the microfluidic channel (e.g., ceiling of the microfluidic channel) may gradually slope and/or curve up/down, or in some spatial combination (i.e., depth and/or width of the microfluidic channel increases or decreases), resulting in a microfluidic channel that gradually becomes shallower and/or deeper, or gradually becomes wider and/or narrower, or combinations thereof. Slopes and curves with diverse ranges of angles and radii of curvature can be utilized. Alternatively, the slope and/or curve of the channel can be changed through multiple steps, where fine step changes can have equivalent function as a gradually changing sloped structure.

Thus, in some embodiments, the microfluidic platform further includes microfluidic channels connecting each chamber of the microfluidic platform. In some embodiments, the microfluidic channels are sloped. In some embodiments, at least one of an X-, Y-, or Z-direction side of the microfluidic channels have a slope that can include, without limitation, up, down, and combinations thereof. In some embodiments, at least one of an X-, Y-, or Z-direction side of the microfluidic channels slope has a spatial combination. In some embodiments, at least one of the microfluidic channels become at least one of shallower, deeper, wider, narrower, or combinations thereof in relation to a first point of the microfluidic channel and a second point of the microfluidic channel.

The constant-height structure is enabled by using microfabrication processes such as additive manufacturing (e.g., three-dimensional (3D) printing), photolithography, electron-beam lithography, stereolithography, X-ray lithography, and ion beam lithography. The curved and/or sloped structure is enabled by using a microfabrication process called two-photon photolithography (2PP). A microfabrication technique using direct laser writing can also be utilized. In the case of 2PP, the method takes advantage of two near-infrared photons to induce polymerization of ultraviolet (UV) photosensitive materials. The precise femtosecond laser scanning throughout the photosensitive material results in a 3D volume being affected whose features can be in the range of few tens to few hundred nanometers, depending on the laser features utilized. The extinction coefficient (magnitude of transition) of this laser-induced photon is below the necessary energy to induce polymerization unless another photon strikes the molecule during the extinction coefficient lifetime (hence the name, two-photon photolithography). 2PP enables precise control in the Z-direction features that can be created, which supersedes that of any other micro- or nano-scale fabrication methods currently available. This aspect provides a unique opportunity, exploited for the first time, to produce multi-functional microstructures used for microfluidic purposes. Alternatively, grayscale lithography may be used. However, such methods have less control over the type of micro and/or nano structures that can be created.

In some embodiments, the microfluidic structures in the present disclosure are created using this 2PP, 3D mask-less lithography process. In a particular embodiment, a Nanoscribe Photonic Professional GT tool (Nanoscribe GmbH) was utilized. This microfabrication tool enables precise control over 2PP fabrication enabling the manufacturing of the aforementioned sloped structures to be integrated within or with microfluidic devices. In some embodiments, the Nanoscribe-fabricated structures were embedded within a previously fabricated microstructure using other microfabrication methods. In other embodiments, the Nanoscribe-fabricated structures were directly printed within previously fabricated microstructures. In other embodiments, the Nanoscribe-fabricated structures were used as a mold to create negative-replicas using appropriate polymers as the microfluidic channel.

The integrative stacking process utilized to align and fabricate multi-layer fluidic systems that have true 3D features is a newly developed process that allows for infinite stacking of adjacent layers composed of sloping structures. This results in microfluidic devices that have desirable, complex channel features of any combination of sloping and/or curving in all spatial orientations simultaneously, which allows for the fabrication of unique 3D channel features.

In various embodiments, aspects of the present disclosure pertain to systems and methods that miniaturize and automate most of the steps of the XPORT DNA transfer process and can be used to transfer natural or synthetic DNA from donor cells into target cells for genetic modification and gene editing. The droplet microfluidic design that automates these assays were designed so that the system is compatible with existing droplet microfluidics-based high-throughput screening systems so that its functions can be further expanded and/or integrated with other systems as needed based on particular applications. The time saving is envisioned to be over 6000-fold higher compared to conventional well plate or culture flask-based assays, and a corresponding reagent consumption saving of at least 100-fold.

Currently, there are no commercially available technologies or techniques for the automation and high-throughput optimization of DNA transfer from donor cells into broad ranges of host cells. In addition, embodiments of the present disclosure do not require special preparation of DNA recipient and/or host cells, such as chemical or electrical competency. No technology exist that is capable of performing DNA transfer by enclosing molecules and living cells in adjustable microscale volumes to maximize DNA transfer efficiency and screen for strains and conditions where such maximum efficiency can be obtained. The combination of incorporating these functionalities (e.g., microdroplet generator, microdroplet merging, microdroplet detection, and microdroplet sorting) to enable the specific application of enhancing DNA transfer between a donor and target cells has not been demonstrated before. In addition, the customizable aspects, such as droplet co-cultivation time that controls the duration of donor and/or target interaction, sorting target (e.g., XPORT-transferred DNA fluorescence in droplet) and donor cell to target cell ratios in a droplet, with respect to the purpose of the device (enable a high efficiency, high throughput XPORT-mediated DNA transfer in microdroplets) is novel.

FIG. 2 shows the basic principles of conjugation applied to the XPORT system. The simplified schematic shows a donor cell excise the payload gene cassette (the genetic region of interest to be delivered to the receiver cell), and, upon cell to cell contact, transfer the gene cassette to the receiver cell which then integrates into the genome of the receiver cell. The bottom portion of FIG. 2 shows the mapped gene region being transferred from donor to receiver.

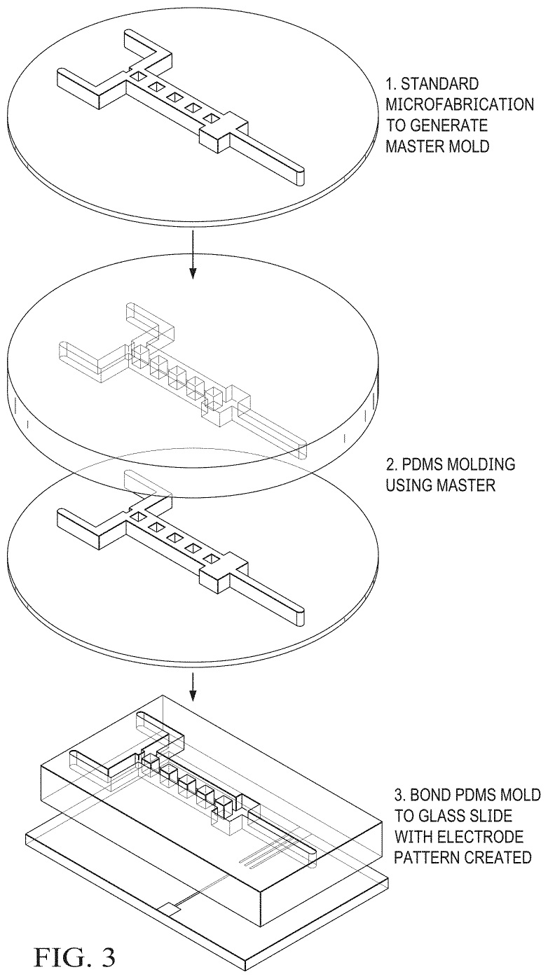

FIG. 3 shows a simplified fabrication process according to an aspect of the present disclosure. However, it should be noted that there are many macro-, micro-, nano-fabrication techniques that can be applied to generate the devices of the present disclosure as described above. PDMS, illustrated in FIG. 3, is just one of many different potential materials that can be used for the devices of the present disclosure. In some embodiments, various polymer materials such as but not limited to polydimethylsiloxane (PDMS), thermoplastic, polymethyl methacrylate (PMMA), polycarbonate (PC), polyethylene (PE), polypropylene (PP), or even glass.

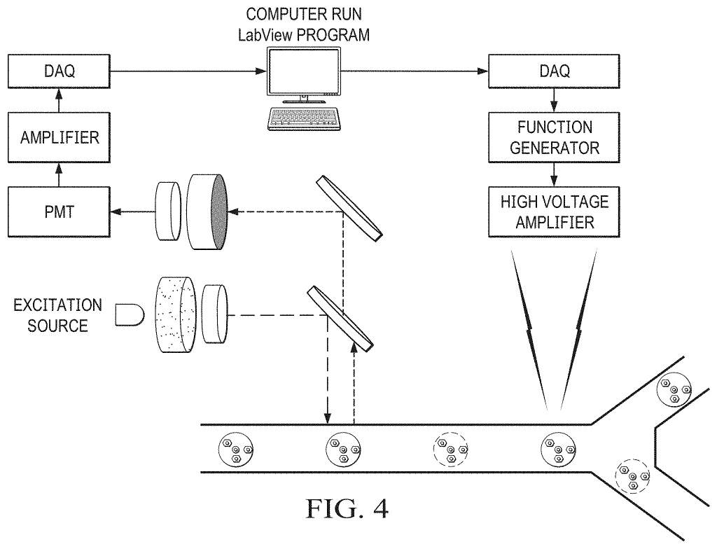

FIG. 4 shows a diagram simplifying how DEP droplet sorting is achieved following an example detection mechanism (in this case, fluorescence detection) of the droplet content. The presented droplet content detection and sorting system includes interchangeable equipment, and only serves as an embodiment according to one aspect of the present disclosure. It should be noted that this embodiment uses DEP sorting as the droplet sorting mechanism and fluorescence as the droplet content detection mechanism. In some embodiments, several alternatives are readily envisioned. In this figure, a photomultiplier tube labelled “PMT” detects fluorescence signal in a volumetric detection region through the capture of emitted photons from fluorophores excited by a laser or light emitting diode (LED)-based excitation source. The PMT converts the fluorescence signal into a current that is passed through an operational amplifier labelled “Amplifier” to a data acquisition board labelled “DAQ” that feed into a custom-written LabVIEW program to inform a waveform function generator labelled “Function Generator” to produce an electric field. This electric field may connect directly to the droplet sorting functionality in the microfluidic device to produce a sorting event. In other embodiments, the Function Generator is serially connected to a high voltage amplifier to propagate an amplified electrical signal which is then connect to the microfluidic droplet sorting functionality.

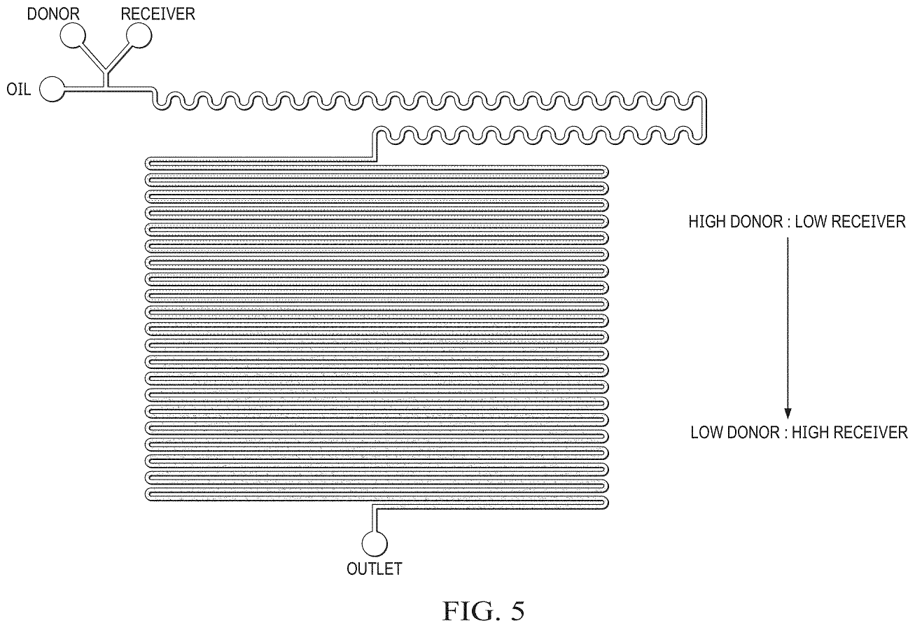

FIG. 5 shows a diagram according to an embodiment of the present disclosure of how to achieve the pairing of a microfluidic gradient generator with a microfluidic droplet generator such that donor cell to target cell ratios inside the generated droplet span an intended range within a single experimental trial. The input labelled “Donor” and “Receiver” can be manipulated using distinct syringe pumps or pressure-drive pumps in which one input starts at a high flow rate and gradually decreases to a lower flow rate while the other input starts at a low flow rate and gradually increases to a higher flow rate, changing the ratio of the donor and receiver cells being co-encapsulated into the generated microdroplet.

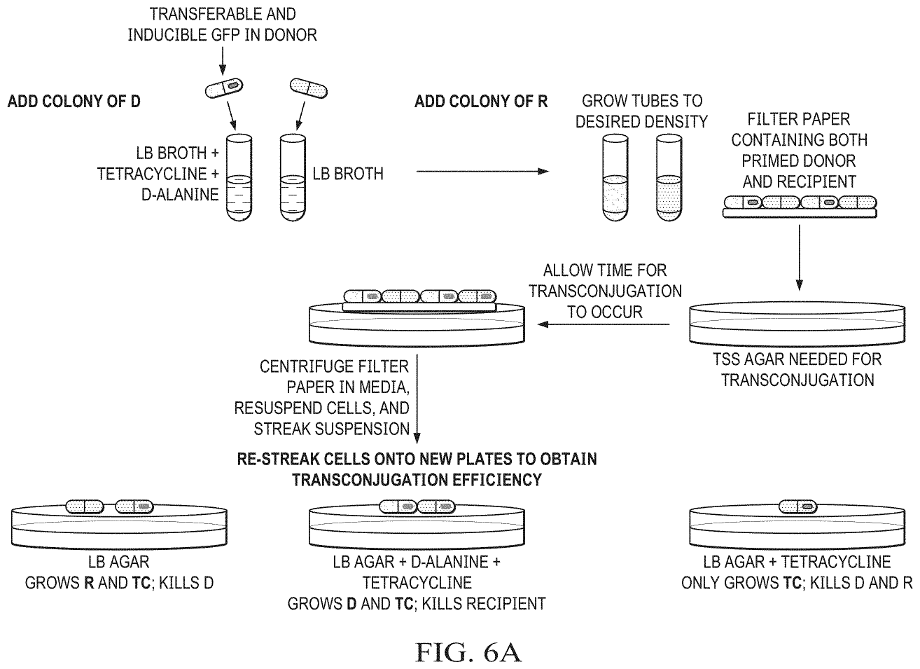

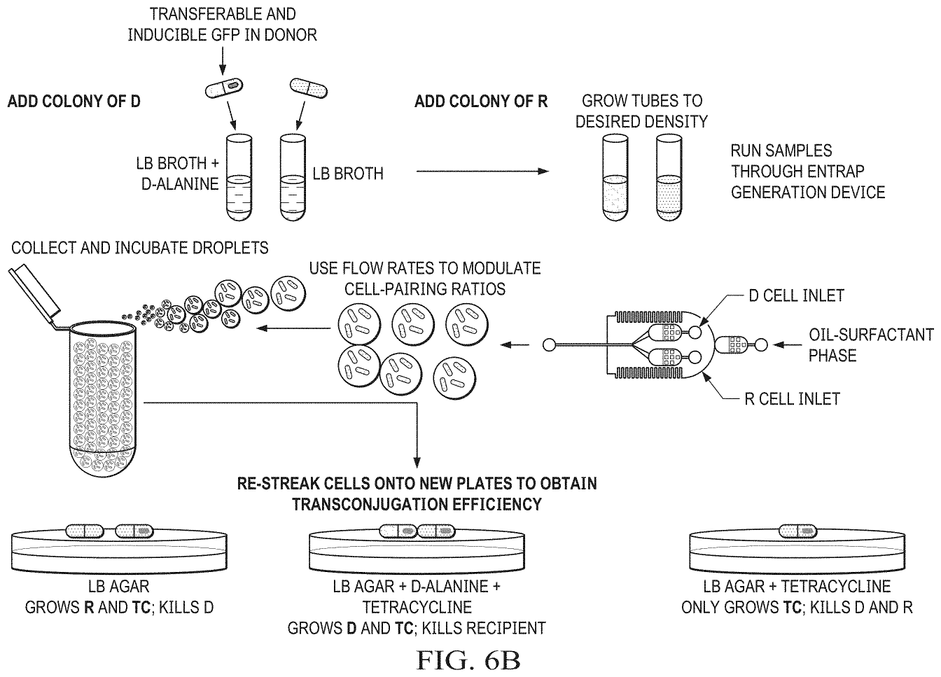

FIGS. 6A-6B shows an overview schematic comparing a conventional XPORT DNA transfer approach (FIG. 6A) and one example configuration of the DNA ENTRAP microfluidic approach (FIG. 6B).

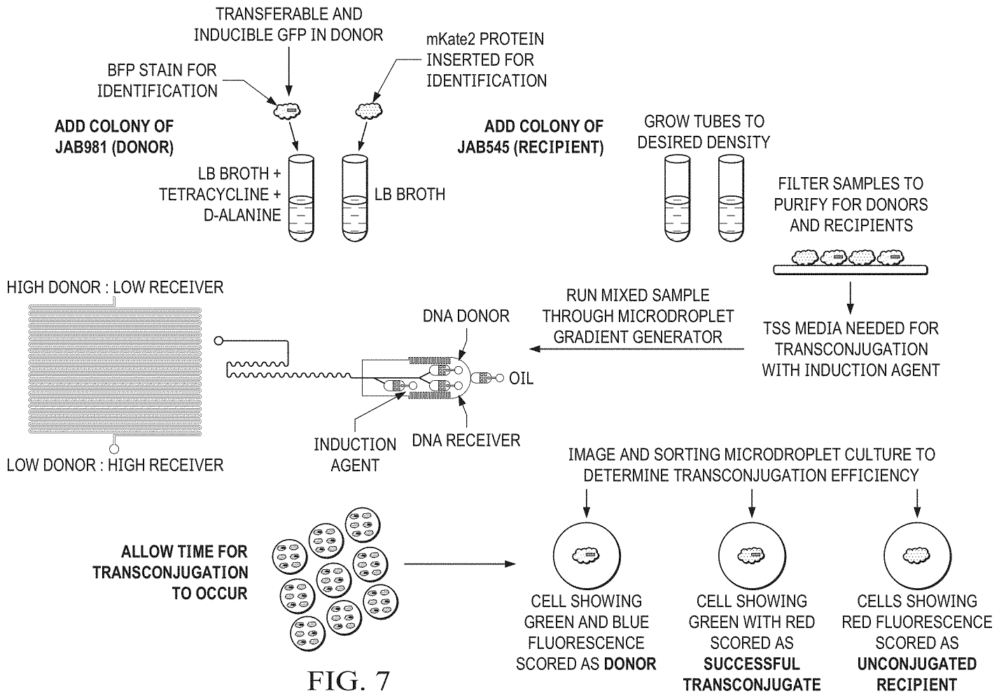

FIG. 7 illustrates a potential alternative workflow of the DNA ENTRAP microfluidic system. Ratiometric cell type pairing is provided here as an example of potential facile experimental condition modulations enabled by the devices of the present disclosure. FIG. 7 details an alternative DNA ENTRAP workflow that includes an in-droplet DNA, RNA, nucleic acid, cell, or other genetic material transfer event detection along with an automated imaging and sorting functionalities. The workflow simplified in FIG. 7 labels the two donor and receiver cell or particles populations labelled with constitutive fluorescent protein reporters or with fluorescent detection markers in order to facilitate rapid and in-droplet identification of cell type.

Taken together, these illustrate that the devices of the present disclosure can run high-efficiency, high-throughput, and automated genetic modification and engineering of non-traditional bacterial hosts, including many hosts that would otherwise not be accessible to genetic modification and gene editing.

Accordingly, in a further embodiment, the present disclosure pertains to a method for cell-to-cell DNA, RNA, or other generic material transfer through use of a water-in-oil emulsion microdroplet-based microfluidic platform (e.g., a DNA ENTRAP device/system) for automation and high throughput identification or screening of genetic transfer outcomes. In general, the method includes adding donor cells and target cells to a microfluidic platform thereby forming droplets, adding an induction media to the microfluidic platform, adding a signal amplification media to the microfluidic platform, detecting functionality to allow for analysis of inner content of the droplets, and sorting the droplets to allow for separation of the droplets based on the analysis of inner content of the droplets. In various embodiments, other potential avenues, such as two-phase emulsions, oil-in-water, hydrogel beads in oil, hydrogel beads in water, water-in-oil-in-water double emulsions, can be utilized as opposed to water-in-oil emulsion avenues.

While the various embodiments disclosed herein utilize DNA, it should be noted that various other applications for the devices of the present disclosure are readily envisioned. For example, in some embodiments, the devices of the present disclosure can be utilized in DNA, RNA, cellular, or small molecule applications. In some embodiments, the devices of the present disclosure can be utilized with particles that can include, without limitation, cells having DNA, cells having RNA, small molecules, and combinations of the same and like.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

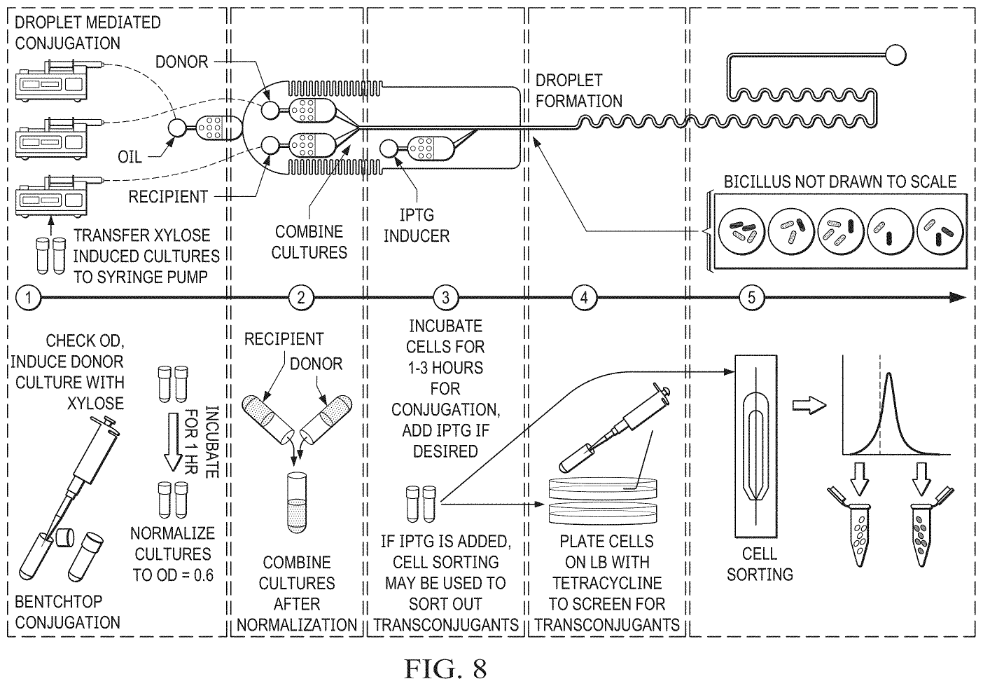

DNA ENTRAP is presented herein as a system to simplify, enhance, and increase the throughput of XPORT-mediated DNA transfer in B. subtilis using a droplet microfluidic approach. It should be noted that the DNA ENTRAP systems of the present disclosure are not limited to use with the XPORT system. Workflows and devices were designed to address the limitations and inefficiencies encountered when attempting to genetically engineer novel chassis, including uncharacterized environmental microbes. Engineered B. subtilis strains were utilized in order to demonstrate the feasibility and efficiency of the DNA ENTRAP systems disclosed herein. These systems achieved rapid and controlled conjugation efficiencies that surpass traditional, benchtop conjugation approaches (FIG. 8).

For the droplet microfluidics approach, FIG. 8 (top panel), xylose-inducted DNA donor cultures and recipient cultures are loaded into dispensing syringes actuated through syringe pumps. The two cultures are combined as the channels converge to form a co-culture solution that then travels to the droplet formation region of the system. The co-culture solution is then formed into water-in-oil emulsion microdroplets that travel through a microfluidic mixing region to mix the inner contents of the droplets. The droplets then travel to a droplet incubation chamber and allowed to co-incubate for 1-3 hr. During a conventional benchtop approach, FIG. 8 (bottom panel), the DNA donor and recipient strains are grown in their preferred medium followed by an induction growth period where the DNA donors are cultured in the presence of xylose. Following this, both cultures are collected and diluted to an optical density (OD) of 0.6. The newly prepared cultures are combined at the experimental ratios (1:1 or 3:1 donor to recipient) and co-incubated in supplemented lysogeny broth (LB) media. The co-cultured solutions are plated onto LB-agar supplemented with tetracycline to screen for the presence of transconjugates. The platforms of the present disclosure achieve a 2-20-fold increase in cell conjugation efficiency depending on the input cell ratio along with the selected incubation duration.

In further detail, FIG. 8 shows a schematic comparing a traditional benchtop approach (bottom) to an example workflow of the DNA ENTRAP system (top) to demonstrate the compatibility of the DNA ENTRAP system with liquid handling systems, illustrating that a fully automated assay using the microfluidic workflow is possible. For droplet conjugation (depicted in the upper half) step 1, xylose-induced donor cultures and recipient cultures are loaded into syringe pumps. In step 2, the two cultures are combined as the channels converge. In step 3 (optional step), an isopropyl β-D-thiogalactopyranoside (IPTG) inducer is added in advance to induce green fluorescent protein (GFP) expression in transconjugants after a successful conjugation event has occurred. In step 4, the co-culture is encapsulated in a water-in-oil droplet at a flow focusing point, where the oil phase pinches off an aqueous droplet. In step 5, droplets are collected into a microcentrifuge tube and incubated to undergo conjugation. For benchtop conjugation (depicted in the lower half), step 1, donor culture and recipient cultures are grown in 3 mL LB (tetracycline 10 μg/mL, D-alanine 100 μg/mL) and LB (with no antibiotics), respectively. Donor strain is induced with 1% xylose at OD between 0.12 to 0.2. Both cultures are incubated for another hour after xylose is added. In step 2, both cultures are collected and diluted to an OD of 0.6, then combined into a single tube at a 3:1 or 1:1 ratio. In step 3, the co-cultures are incubated 1-3 hr in LB (with D-alanine), and may be induced with IPTG if desired. If IPTG is added, then in step 5, the transconjugants may be directly sorted out using a cell sorter for induction of GFP expression. Otherwise, in step 4, the co-cultures are plated on a selective media.

Taken together, these results establish DNA ENTRAP as a powerful technology for rapid XPORT-mediated genetic engineering, enabling both high speed screening for cell compatibility and parameter optimization (e.g., cell ratio and co-incubation length). Studies of high-throughput (e.g., —2.5 assays/s) B. subtilis XPORT conjugation via DNA ENTRAP demonstrate that it has the capability to genetically engineer microbes to maximize their highly diverse biotechnological potential in synthetic biology.

Discussion Efficient XPORT-mediated conjugation requires extensive tuning of growth parameters, strain growth state, and other pre-determined physiological conditions that impact the ability of conducting conjugative DNA transfer. In order to circumvent these hurdles, DNA ENTRAP was developed. DNA ENTRAP aims to be a tool in the future synthesis, discovery, and development of novel organisms. The present disclosure demonstrates conjugative DNA transfer using an XPORT Bacillus ICE system within a droplet microfluidic system, a novel platform capable of conducting a highly customizable and tunable gene transfer assay that enables percent increases ranging from 200% to 13,000% depending on the selected assay parameters.

As the first step in demonstrating the devices of the present disclosure, studies utilizing the XPORT system as a newly established genetic tool were conducted. One utility of the platforms described herein is in conducting massively parallel synthetic engineering of environmental isolates via XPORT. To this end, the devices and protocols of the present disclosure are envisioned to transition into screening environmental isolates. Furthermore, the capability in screening and analyzing highly complex cellular libraries with an abundance beyond the screening capability of benchtop researchers are realized with the platforms of the present disclosure. Even at a modest droplet functionalities throughput of 10s of droplets per second, the DNA ENTRAP systems of the present disclosure can screen a population size of 10⁶ environmental isolates in under 4 days. Using the conventional, agar-plate based strategy and approach, with a day and night shift team of 25 scientists working 12 hours shifts each (50 plates/day), the same 10⁶ environmental isolate population can be screened in 54.8 years. In order to fully realize this goal of full-platform automation, the development of system hardware that incorporates similar detection and sorting systems that have demonstrated functionality and utility are envisioned. Through the biological mechanisms demonstrated herein, a complete platform that aligns with the need of improving microfluidic technology by providing a powerful, automated, high-throughput, and high reliability system are offered.

Despite the many advantages, the current status of the DNA ENTRAP platform offers several interesting avenues of advancement. While the systems and methods established the potential for DNA ENTRAP to enhance genetic material transfer efficiency, some embodiments of the presented disclosure were limited to two B. subtilis strains, a single donor B. subtilis and a single receiver B. subtilis strains. Given the extent of previous demonstrations of XPORT-mediated DNA transfer into environmental isolates and other non-Bacillus species, it is envisioned that the developed platforms and techniques presented herein can be utilized to introduce foreign DNA into a wide range of prospective recipients. Additionally, culturing and scaling inconsistencies with growing the engineered XPORT strain resulted in a longer than expected assay development time to establish a results-driven link between off-chip and on-chip methods. It is envisioned that a more robust, engineered donor strain could overcome some of these variability issues. A success read-out for in-droplet conjugation is desired for the final platform. By incorporating this strategy, the possibility for a droplet sorting modality to be integrated into the platforms of the present disclosure is enabled. These engineering aspects enhance DNA ENTRAP as a transformative genetic transfer tool.

As illustrated herein, the DNA ENTRAP platforms of the present disclosure provide enhanced and automated conjugative events with respect to traditional benchtop approaches. These platforms conduct the previously unrealized scientific feats of using an XPORT-conditioned donor Bacillus subtilis strain to transfer DNA to recipient cells while encapsulated within microdroplets. This systems and methods of the present disclosure help overcome the limitations of traditional genetic manipulation and DNA transfer approaches by confining the cells in nano-sized water-in-oil emulsions to encourage cell-to-cell contact, which is needed for conjugation to occur.

Reference will now be made to particular materials and methods utilized by various embodiments of the present disclosure. However, it should be noted that the materials and methods presented below is for illustrative purposes only and is not intended to limit the scope of the claimed subject matter in any way.

Device Fabrication. DNA ENTRAP is designed to harness the spatial and temporal qualities unique to droplet microfluidics to enhance DNA transfer events. The system is designed to perform the following basic functions: (1) generation of large number of water-in-oil-emulsion droplets encapsulating donor and recipient cell candidates, each serving as independent nano-liter scale bioreactors; (2) incubation of the cell-encapsulated droplets for varying times; (3) facile ratiometric cellular encapsulation screening; and (4) enablement to pairing with automation modalities, that is during choosing the specifics of the workflow a consideration and preference is set for those techniques that can be later fully automated.

As mentioned earlier, the microfluidic systems that integrates all of these functional steps is capable of fully automated operation through a LabVIEW-controlled custom interface to address the needs of certain cellular species, allowing for a continuous operation of DNA ENTRAP with minimal human intervention. This minimization of manual sample and reagent handling steps, which improves the biological integrity and reduces the possibility of human error, enhances the overall platform stability. Each of the functional components of the DNA ENTRAP device, namely cell-encapsulated droplet generation, incubation of droplets, detection of droplet contents using an optical setup (currently based on microscopic imaging, with flow-through fluorescence detection further envisioned), follow well-established techniques and instrumentation configurations widely utilized in the field of droplet microfluidics.

Various aspects of the DNA ENTRAP were analyzed and characterized in order to verify the platform stability and performance. The platform produced uniform cell-encapsulated 60 μm diameter droplets containing Bacillus subtilis cells. Then, starting in-syringe cellular ODs were characterized to achieve the desired in-droplet cell concentration ranges. Finally, droplet generation rates were optimized in order to ensure tightly controlled droplet incubation time ranges. The overall assay speed (considering a single droplet being generated) was in the range of 180-200 droplets/sec. Devices used in this study were fabricated using standard PDMS soft lithography approaches.

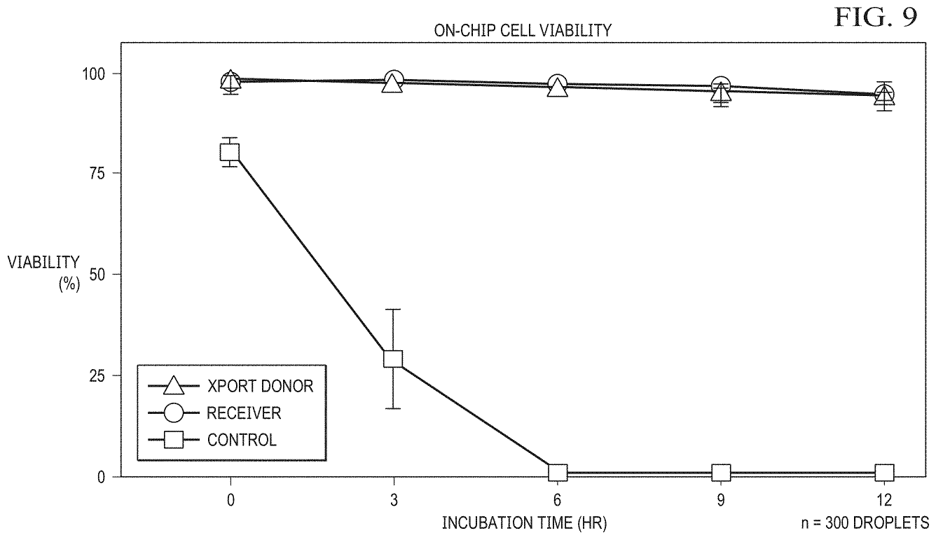

Droplet Cell Viability. To check the cell viability of the Bacillus s. JAB 981 DNA donor strain and Bacillus s. JAB 545 recipient strain in droplets, cells were encapsulated in droplets that contained YOYO™-1, a compound that selectively fluorescently stains dead cells. Approximately 25-30 cells were encapsulated per droplet and viability was monitored for 12 hr, the maximum expected duration of the entire in-droplet assay. First, each strain was encapsulated in a droplet and tagged with a Hoescht blue fluorescent probe to enable cell identification. Over the 12 hr analysis period, neither the JAB 981 or JAB 545 populations displayed any appreciable loss of viability (>96% viability) when encapsulated in a droplet. Over the 12 hr analysis period, neither strain demonstrated any loss of viability when co-encapsulated in a droplet (>96% viability). In order to confirm the droplet functionality and compatibility of YOYO™-1 fluorescent stain to identify dead Bacillus s. cells, the JAB 545 strain was encapsulated in droplets containing 10 μg/mL tetracycline for 6 hr. The JAB 545 stains showed viabilities of 80%, 40%, and <5% at the 1 hr, 2 hr, and 3 hr post encapsulation timepoints, respectively. FIG. 9 shows experimental results comparing XPORT donor cell viability over the course of 12 hours in droplets and receiver cell viability over the course of 12 hours in droplets. Standard deviation denoted by error bars obtained from triplicate experiments.

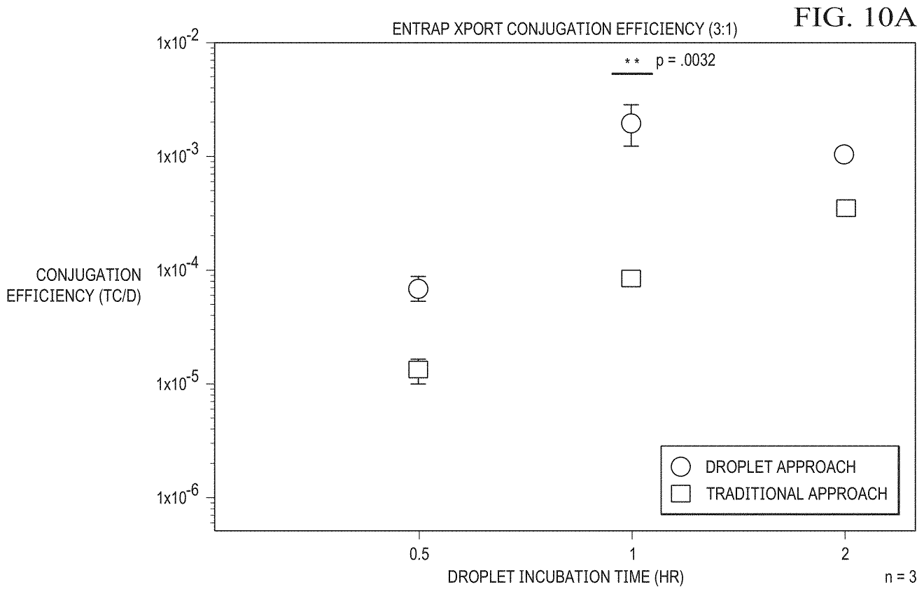

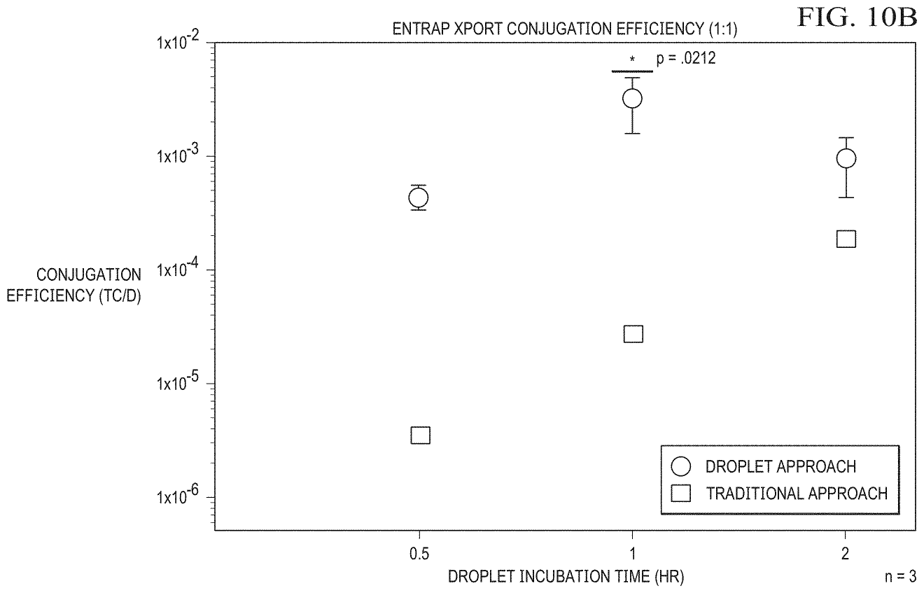

Droplet-Based Conjugation. To confirm whether the underlying technology of DNA ENTRAP is capable of performing the relevant biological assays, cells were co-encapsulated in droplets at two different ratios, 3:1 and 1:1 donor cells to recipient cell ratios, respectively, for 0.5 hr, 1 hr, and 2 hr co-incubation periods. Conjugation efficiency (TC/D) is determined by the ratio between the number of donor (D) colonies to the number of transconjugate (TC) colonies on their respective selection media. This ratio is of particular interest as it provides the most accurate metric to compare the donor strain's capability to perform gene transfer events across various culture conditions. The TC/D was determined to be 6.95×10⁻⁵, 1.93×10⁻³, and 1.06×10⁻³ for a 3:1 ratio at 0.5 hr, 1 hr, and 2 hr, respectively. The conjugation efficiency was determined to be 3.80×10⁻⁴, 3.49×10⁻³, and 9.46×10⁻⁴ for a 1:1 ratio at 0.5 hr, 1 hr, and 2 hr, respectively (FIG. 10A and FIG. 10B). When compared to the traditional approach of 1:1 DNA donor to recipient cell ratio, statistical significance reinforcing superiority of the droplet microfluidics approach compared to the traditional approach was observed for the 1 hr co-incubation condition (p-value of 0.0212). Likewise, when the 3:1 DNA donor to recipient cell condition was analyzed, a similar statistical superiority of the droplet microfluidics approach compared to the traditional approach was seen for the 1 hr co-incubation condition (p-value of 0.0032). Additionally, when time conditions between the different experimental conditions were examined for significance for both DNA donor to recipient ratios, the droplet microfluidics approach data points for the 0.5 hr and 1 hr demonstrated a significant difference (p-value of 0.0050 for the 3:1 ratio and p-value of 0.0284 for the 1:1 ratio). For all statistical analyses, a two-way analysis of variance (ANOVA) assuming sphericity with an alpha of 0.05 and a Šídák's multiple comparison test was conducted to obtain an adjusted p-value. The Šídák's method was preferred to the Bonferroni method as it provides more power to the calculated p-value.

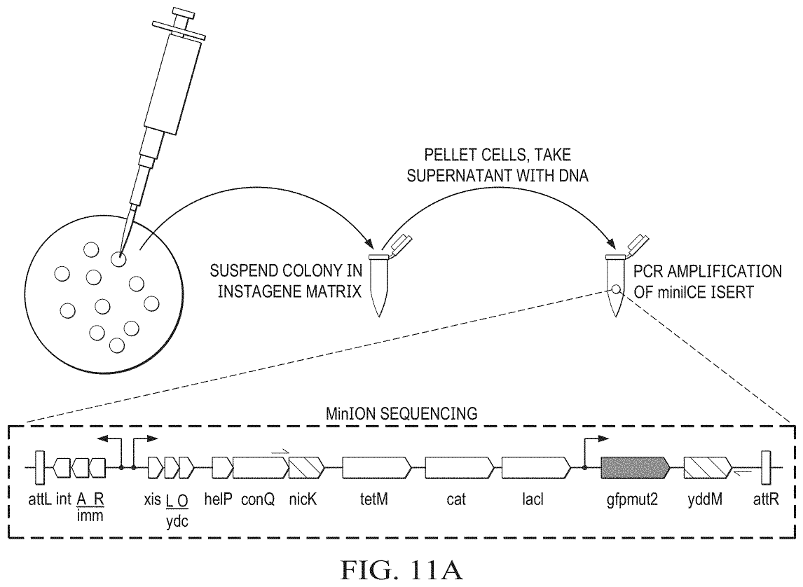

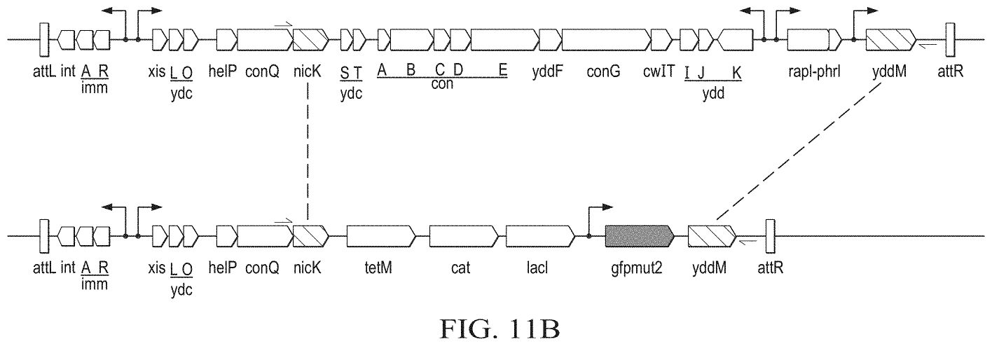

Conjugation Confirmation Assays. Genetic Confirmation and Population-Based Sequencing: Positive transconjugates resulting from XPORT-mediated conjugation should contain the IPTG-inducible GFP cassette in addition to a tetracycline resistance marker, integrated between the nicK and yddM gene loci on the recipient genome. Colony polymerase chain reaction (PCR) amplification was performed on droplet transconjugates to confirm the insertion of the miniICE gene cassette into the genome. The amplified DNA fragment was further sequenced to confirm the validity of the transferred DNA. FIG. 11A shows the workflow schematic for the confirmation assays, with the depiction of the DNA transferred from the DNA donor cell to the receiver cells shown in FIG. 11B.

In order to confirm the location of the miniICE gene cassette in the recipient genome and the fidelity of the insertion, full genome sequencing on a population of transconjugants was performed. From the passed long reads (roughly 2.4 million reads per replicate), a total of 522 reads were found to match at least 150 bp of the miniICE region. These reads were extracted and mapped to the expected miniICE region in the recipient. The consensus alignment demonstrated successful integration of the miniICE cassette at the tms-leu2 gene locus. Results from the sequencing demonstrating successfully target loci overlap.

Flow Cytometry Confirmation for Transconjugant Induction: Transconjugants were selected from LB (tetracycline¹⁰ μg/mL) plates and subsequently inoculated in LB broth with tetracycline. Upon reaching an OD of ˜1.0, transconjugants were induced with 1 mM IPTG for 1 hour. After induction, 5 μL of culture was diluted into 500 μL phosphate buffered saline (PBS) for flow cytometry analysis. Droplet transconjugants showed comparable fluorescence intensities with benchtop transconjugants. Fluorescence-activated cell sorting confirmed the droplet transconjugant (JAB 545 transconjugant) performs similarly to the positive control (JAB545 benchtop transconjugant).

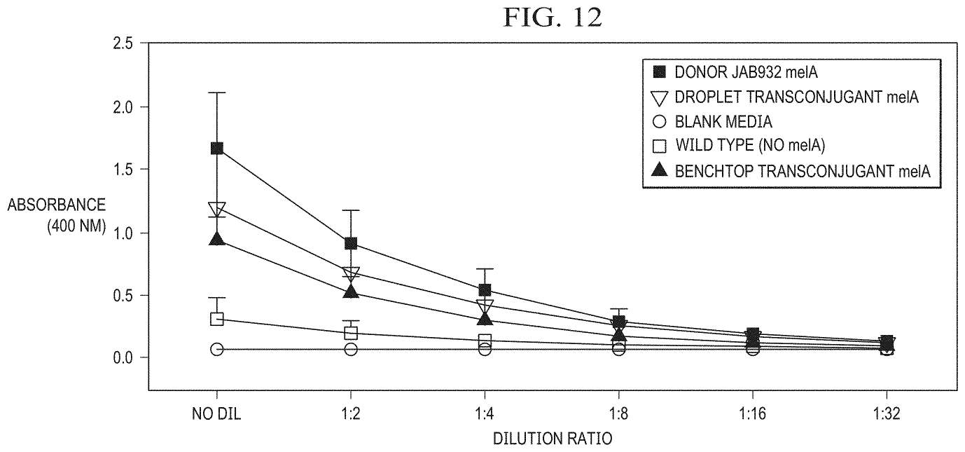

DNA ENTRAP for Melanin Production by Transconjugated Cells. To demonstrate the utility of the DNA ENTRAP system in the conjugation of other gene cassettes, the donor strain was reengineered to confer a tyrosinase gene to a recipient Bacillus subtilis. The tyrosinase gene, derived from Rhizobium etli, facilitates the conversion of tyrosine to 3,4-dihydroxyphenylalanine (DOPA) and to dopaquinone. Subsequent oxidation steps lead to the formation of melanin, a biomolecule that has implications for UV protection and therapeutics. The melanin-producing transconjugants and their respective benchtop transconjugant controls were cultured and supernatants were collected after 3 days. No significant difference was found in absorbance readings for melanin between the droplet and the benchtop transconjugants (FIG. 12).

Device Fabrication. As disclosed herein, devices that are proficient in evaluating XPORT media DNA transfer using microdroplet technology is demonstrated. DNA ENTRAP is fabricated using standard PDMS-based soft lithography techniques. The droplet microfluidic devices used for performing the aforementioned is composed of two functional components on separate, but tethered chips (droplet generation and droplet incubation). Droplet generation (diameter 60 μm) is accomplished using a single cross junction droplet generator composed of a horizontal carrier oil channel (55 μm wide) and a perpendicular water channel (50 μm wide), both with a height of 65 μm. A droplet culture and observation device are used to assess the cell viability and co-encapsulation cell/droplet numbers using Zeiss Colibri Axiovert 200 optical microscope. The device exploits 400 μm×400 μm pillars spaced 20 μm apart, serving as an in-house designed massive basket-like trapping chamber for a monolayer of droplets to be collected, analyzed, and observed within a microfluidic chamber (10 mm×3 mm). The height of the culture chamber was set to 100 μm to allow for single droplet layer observation of 60 μm droplets while ensuring minimal pressure buildup. The system uses a syringe-based flow driven pump and medical grade tubing for transferring droplets between tethered devices. The inner two inlets are used to introduce cells and the perpendicular channels were used to introduce fluorinated oil (NOVEC™ 7500, 3M™) with surfactant (2.5 wt/wt %, PICO-SURF™ 1, Dolomite Microfluidics, MA) in order to generate and stabilize the microdroplets.

For obtaining 100 μm thick microchannel patterns, a master silicon wafer was spin-coated with negative photoresist (SU-8 2075, Microchem Corp., MA) at 2100 rpm, followed by soft-baking in two steps at 65° C. and 95° C. for 24 hr and 20 min, respectively. Following the soft-bake, the master was exposed to UV light using a standard photolithography mask aligner (EVG 610, EVG Group, Germany) and post-exposure baked at 65° C. and 95° C. for 40 min and 20 min, respectively. After developing the unexposed photoresist (EBR 10A, Microchem Corp., #10018079, MA), the patterned wafer was coated with (tridecafluoro-1,1,2,2 tetrahydrooctyl) trichlorosilane (United Chemical Technologies, Inc., Bristol, Pa.) for 20 min using a conventional desiccator, to prevent pattern removal during PDMS replication. For obtaining 65 μm thick microchannel patterns, a master silicon wafer was spin-coated with negative photoresist (SU-8 2050, Microchem Corp., MA) at 2600 rpm, followed by soft-baking in two steps at 65° C. and 95° C. for 24 hr and 20 min, respectively. Following the soft-bake, the same master mold preparation procedure was carried out as described for the completion of the 100 μm height master mold. A thin, 30 μm thick, PDMS (SYLGARD® 184, Dow Corning Corp., MI) layer was spin-coated on the patterned glass slide at 3000 rpm for 30s to obtain a hydrophobic bottom surface that is necessary for droplet generation. For both devices, a PDMS microchannel was replicated from the master by pouring 20 g of PDMS mixture (1:10 curing agent to polymer) onto the master secured in a plastic petri dish. After polymerization, the merging device was bonded to the PDMS-coated glass slide using conventional oxygen plasma treatment (Plasma Cleaner, Harrick Plasma, Ithaca, N.Y.) for 90 s. After polymerization, the droplet generator and culture chamber microchannels were bonded to a PDMS-coated glass slide (Micro Slides 2947-75×50, Corning Inc., NY) using the same oxygen plasma treatment protocol. All PDMS microchannels were filled with a commercial hydrophobic surface coating agent (1H, 1H, 2H, 2H-perfluorododecyltrichlorosilane, Sigma-Aldrich, MO) mixed with fluorinated oil (NOVEC™ 7500, 3M™) at 1 wt/wt % and flushed prior to use. The fabrication of the DNA ENTRAP system illustrates the development of a fully automated, high-throughput DNA enhancing platform.

Droplet Cell Viability. The cellular components of the system were analyzed for microdroplet cell viability using a two-step approach. First, donor strain cell (JAB 981) viability was demonstrated and characterized in a droplet culture. For these experiments, a colony was grown from a recently (<14 days old) streaked plate and grew the Bacillus s. JAB 981 strain in LB media supplemented with 100 μg/mL D-Alanine and 10 μg/mL tetracycline for three hours in 3 mL total supplemented media volume until culture reached an OD600 of 0.95-1.10. Following, 2.5 mL of the culture was centrifuged at 3000 relative centrifugal force (RCF) for 5 min and washed with PBS twice repeating the pelleting process between each wash step. The cells were suspended in 500 μL sterile PBS and a blue Hoescht 33342 fluorescent stain was used to label the cells for in-droplet identification. Following this staining period, the cells were centrifuged, wash 2×, and then resuspended in 500 μL fresh sterile PBS. The cells were then added to 3 mL fresh 1× transformation and storage solution (TSS)/LB media containing 100 μg/mL D-alanine and 1 μM YOYO™-1 iodide cell impermeant, dead-cell fluorescent stain. The suspension was then used to generate droplets of 60 μm in diameter. Fluorinated oil (Novec 7500, 3M) with surfactant (2.5 wt/wt %, PICO-SURF™ 1, Dolomite Microfluidics, MA) in order to generate and stabilize the microdroplets. The droplets flowed from the droplet generator device into the culture device and were monitored over 12 hr using fluorescent microscopy to distinguish between the live and dead cells. Second, the intended recipient Bacillus s. JAB 545 strain was assessed using a similar approach. A JAB 545 colony was picked and cultured in LB media for three hours until the culture reached an OD600 of 0.95-1.10. Following, 2.5 mL of the culture was centrifuged at 3000 RCF for 5 min and washed with PBS twice repeating the pelleting process between each wash step. The cells were suspended in 500 μL sterile PBS and a blue Hoescht 33342 fluorescent stain was used to label the cells for in-droplet identification. Following this staining period, the cells were centrifuged, wash 2×, and then resuspended in 500 μL fresh sterile PBS. The cells were then added to 3 mL fresh 1× transformation and storage solution (TSS)/LB media containing 100 μg/mL D-alanine and 1 μM YOYO™-1 iodide cell impermeant, dead-cell green (pseudo-colored red for images) fluorescent stain. The suspension was then used to generate droplets of 30 μm in radius. The droplets flowed from the droplet generator device into the culture device and were monitored over 12 hr using fluorescent microscopy to distinguish between the live and dead cells. Next, individual cell type viability was assessed when during a two-step droplet experiment approach. During the first run, the above process was repeated to encapsulate a total OD600 of 0.6 (0.3 of each cell type) with the JAB 981 donor cells stained with the blue Hoescht 33342 fluorescent stain and incubating the cells prior to droplet generation with 1 μM YOYO™-1 iodide cell impermeant, dead-cell green (pseudo-colored red for images) fluorescent stain. In the next run, the above process was repeated again to encapsulate a total OD600 of 0.6 (0.3 of each cell type) with the 545 recipient cells stained with the blue Hoescht 33342 fluorescent stain and incubating the cells prior to droplet generation with 1 μM YOYO™-1 iodide cell impermeant, dead-cell red fluorescent stain. Finally, a confirmation assay was carried out to confirm YOYO™-1 dead cell staining capability. Briefly, the JAB 545 recipient cells were culture as mentioned above. Immediately following the final suspension in to 3 mL1× transformation and storage solution (TSS)/LB media containing 30 μg of tetracycline was added to the media in order to demonstrate not only the effectiveness of YOYO™-1 at staining dead cells but the ability to distinguish the fluorescent signal over any background LB media fluorescent noise. All experiments were completed in triplicates. Through these assays, the DNA ENTRAP system demonstrated capability of maintaining cellular functionality and it is readily envisioned that this capability extends to a wide variety of contrasting and distinctive cellular species, that is, beyond the cell lines presented herein.

Droplet-Based Conjugation Assays. Single colonies of both the JAB 545 and JAB 981 strains were picked and cultured in LB media and LB+D-alanine¹⁰⁰, respectively. The cultures were set in a shake incubator at 225 rpm and 37° C. for 1.5 hr until the JAB 981 culture reached an OD600 of 0.15. At this time, 60 μL of 50% xylose was added to the 3 mL culture to prime the strain machinery for conjugative element production. The strains were then cultured for another 1.5 hr until OD600 was 0.9-1.1. Following, 2.5 mL of the culture was centrifuged at 3000 RCF for 5 min and washed with PBS twice repeating the pelleting process between each wash step. The cells were suspended in 500 μL sterile PBS. Following, cells were added to fresh LB media supplemented with 100 μg/mL D-alanine to achieve an OD600 of 0.6 at 3:1 and 1:1 donor to recipient OD ratios. For the 3:1 comparison, the donor JAB 981 strain was added to the fresh media until an OD600 of 0.45 was achieved. Next the recipient JAB 545 strain was added to the same media until an end OD600 of 0.60 was achieved. The suspension was then used to generate droplets of 60 μm in diameter. The droplets flowed from the droplet generator device into the designed micro vessel system and incubated for the designated set periods of time before spread plating. For the 1:1 comparison, the donor JAB 981 strain was added to the fresh media until an OD600 of 0.30 was achieved. Next the recipient JAB 545 strain was added to the same media until an end OD600 of 0.60 was achieved. The suspension was then used to generate droplets of 60 μm in diameter. Fluorinated oil (NOVEC™ 7500, 3M™) with surfactant (1.25 wt/wt %, PICO-SURF™ 1, Dolomite Microfluidics, MA) in order to generate and stabilize the microdroplets. The droplets flowed from the droplet generator device into the designed micro vessel system and incubated for the designated set periods of time before spread plating. After the set incubation periods to allow for conjugation to occur, the cultures were removed from the incubator and 50 μL of droplets were pipetted to agar plates and spread. At each timepoint, 50 μL of each test ratio was spread on an LB agar plate to count JAB 545 and successful transconjugates, a LB+D-alanine¹⁰⁰+tetracycline¹⁰ to count JAB 981 colonies and transconjugates, and a LB+tetracycline¹⁰ to select for transconjugates only to determine conjugation efficiency. The experiments were conducted in triplicate over 3 consecutive days.

Droplet-Based Conjugation Assay Efficiency Analysis. In order to accurately account for conjugation efficiency in microdroplets, a selective media strategy was used to shed insight into conjugation event efficiency. During droplet harvesting after the set incubation period, 50 μL of droplets were pipetted to agar plates and spread. LB+D-alanine¹⁰⁰+tetracycline¹⁰ agar plates were used to quantify the number of colony-forming units (CFUs) that could be attributed to both JAB 981 and successful transconjugates. LB agar plates were used to quantify the number of CFUs that could be attributed to both JAB 545 and successful transconjugates. LB+tetracycline¹⁰ agar plates were used to quantify the number of CFUs that could be attributed to successful transconjugates. To get accurate JAB 981 CFUs counts, the number of CFUs counted on LB+tetracycline¹⁰ agar plates were subtracted from the number of CFUs counted on LB+D-alanine¹⁰⁰+tetracycline¹⁰ agar plates. To get accurate JAB 545 CFUs counts, the number of CFUs counted on LB+tetracycline¹⁰ agar plates were subtracted from the number of CFUs counted on LB agar plates.

Conjugation Confirmation Assays. Genetic Confirmation and Population-Based Sequencing: Two primers flanking the insertion site on the recipient genome were used to perform a PCR confirmation of a successful droplet conjugation event. PCR amplification was performed with Q5® polymerase (New England Biolabs), gel purified, and sequenced using the MinION (Oxford Nanopore Technologies).

Flow Cytometry Confirmation for Transconjugant Induction: Both droplet transconjugants and traditional transconjugants (generate via benchtop methods) were struck onto LB (tetracycline¹⁰ μg/mL) and incubated for 16 hours at 37° C. The following day, three colonies from the plate were picked and grown in 3 mL volumes of LB (tetracycline¹⁰ μg/mL) media at 37° C., shaking at 225 rpm. After 2 hr of growth, subcultures were started with an OD600 of 0.05. The cultures were grown to an OD600 of ˜0.5 and induced with 1 mM IPTG. Cultures were shaken at 37° C. for 1 hour. After induction, 5 μL samples were diluted into 500 μL NERL™ Blood Bank Saline for flow cytometry analysis. Flow cytometry was performed on the Sony SA3800 spectral analyzer with the following parameters: threshold forward scatter (FSC) value: 0.5%; FSC gain: 17; side scatter (SSC) voltage: 20%; and fluorescence PMT voltage: 69.8%.

Generating a melA Donor Strain. To construct the donor Bacillus subtilis strain JH642 containing melA, a constitutively expressed melA gene derived from Rhizobium etli was inserted between the nicK and yddM genes of a Bacillus subtilis engineered strain JAB932. The melA gene was cloned under the control of the Pveg promoter paired with the spoVG RBS, both native to Bacillus subtilis. In-droplet and benchtop conjugation were performed in the same way as described for the JAB981 and JAB545 conjugations.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A microfluidic platform comprising:
a droplet generator comprising an entry point for donor particles and target particles;
a first droplet incubation chamber in fluid communication with the droplet generator, wherein the first droplet incubation chamber is configured to allow interaction between the donor particles and the target particles;
a droplet detection functionality to allow for analysis of the inner content of droplets, wherein the droplet detection functionality is at least one of paired with or incorporated on the microfluidic platform; and
a droplet sorting functionality to allow for the separation of droplets based on the analysis of the inner content of droplets, wherein the droplet sorting functionality is at least one of paired with or incorporated on the microfluidic platform.

2. The microfluid platform of claim 1, wherein the droplet generator comprises a first droplet generator for the donor particles and a second droplet generator for the target particles in fluid communication with the first droplet incubation chamber thereby allowing flow of the droplets containing donor particles and the target particles into the first droplet incubation chamber.

3. The microfluid platform of claim 1, wherein the droplet generator comprises a first droplet generator for the donor particles and a second droplet generator for the target particles in fluid communication with the first droplet incubation chamber, and wherein the first droplet incubation chamber comprises separate donor and target particle incubation chambers thereby allowing flow of droplets containing the donor particles into the donor incubation chamber and droplets containing the target particles into the target droplet chamber.

4. The microfluidic platform of claim 1, further comprising:
an induction media or signal-activating media droplet generator comprising an entry point for at least one of an induction media or a supplemental media;
a first droplet merging region in fluid communication with the first droplet incubation chamber, wherein the induction or signal-activating media droplet generator is configured to release at least one of the induction media or the supplemental media at a point between the first droplet incubator and the first droplet merging region; and
a second droplet incubation chamber in fluid communication with the first droplet merging region, wherein the second droplet incubation chamber is configured to allow for signal induction.

5. The microfluidic platform of claim 1, further comprising:
a signal amplification media droplet generator comprising an entry point for signal amplification media;
a second droplet merging region in fluid communication with the second droplet incubation chamber, wherein the signal amplification media droplet generator is configured to release the signal amplification media at a point between the second droplet incubation chamber and the second droplet merging region; and
a third droplet incubation chamber in fluid communication with the second droplet merging region, wherein the third droplet incubation chamber is configured to allow for signal amplification.

6. The microfluidic platform of claim 1, further comprising:
a first outlet, wherein the first outlet is configured to release waste particles displaying no transfer; and
a second outlet, wherein the second outlet is configured to recover particles displaying successful transfer.

7. The microfluidic platform of claim 1, wherein the donor particles and target particles comprise at least one of DNA, RNA, cells, small molecules, or combinations thereof.

8. The microfluidic platform of claim 1, wherein the donor particles and target particles comprise cells comprising DNA.

9. The microfluidic platform of claim 8, further comprising:
a first outlet, wherein the first outlet is configured to release waste cells displaying no DNA transfer; and
a second outlet, wherein the second outlet is configured to recover cells displaying successful DNA transfer.

10. The microfluidic platform of claim 1, further comprising microfluidic channels connecting each chamber of the microfluidic platform.

11. The microfluidic platform of claim 10, wherein the microfluidic channels are sloped.

12. The microfluidic platform of claim 11, wherein at least one of an X-, Y-, or Z-direction side of the microfluidic channels have a slope selected from the group consisting of up, down, and combinations thereof.

13. The microfluidic platform of claim 10, wherein at least one of an X-, Y-, or Z-direction side of the microfluidic channels slope has a spatial combination, and wherein at least one of the microfluidic channels become at least one of shallower, deeper, wider, narrower, or combinations thereof in relation to a first point of the microfluidic channel and a second point of the microfluidic channel.

14. A microfluidic platform comprising:
a droplet generator comprising an entry point for donor particles and target particles;
a first droplet incubation chamber in fluid communication with the droplet generator, wherein the first droplet incubation chamber is configured to allow interaction between the donor particles and the target particles;
an induction media droplet generator comprising an entry point for induction media;
a first droplet merging region in fluid communication with the first droplet incubation chamber, wherein the induction media droplet generator is configured to release the induction media at a point between the first droplet incubator and the first droplet merging region;
a second droplet incubation chamber in fluid communication with the first droplet merging region, wherein the second droplet incubation chamber is configured to allow for signal induction;
a signal amplification media droplet generator comprising an entry point for signal amplification media;
a second droplet merging region in fluid communication with the second droplet incubation chamber, wherein the signal amplification media droplet generator is configured to release the signal amplification media at a point between the second droplet incubation chamber and the second droplet merging region;
a third droplet incubation chamber in fluid communication with the second droplet merging region, wherein the third droplet incubation chamber is configured to allow for signal amplification;
a first outlet, wherein the first outlet is configured to release waste particles displaying no transfer; and
a second outlet, wherein the second outlet is configured to recover particles displaying successful transfer.

15. The microfluidic device of claim 14, wherein the donor particles and target particles comprise at least one of DNA, RNA, cells, small molecules, or combinations thereof.

16. The microfluidic platform of claim 14, wherein the donor particles and target particles comprise cells comprising DNA, wherein the first outlet is configured to release waste cells displaying no DNA transfer, and wherein the second outlet is configured to recover cells displaying successful DNA transfer.

17. The microfluidic platform of claim 14, wherein each droplet generator, each droplet merging region, and each droplet incubation chambers are fluidly connected via microfluidic channels, and wherein the microfluidic channels have a portion comprising at least one of a sloped region, a flat region, or combinations thereof.

18. The microfluidic platform of claim 17, wherein at least one of an X-, Y-, or Z-direction side of the microfluidic channels slope in a spatial combination, and wherein at least one of the microfluidic channels become at least one of shallower, deeper, wider, narrower, or combinations thereof in relation to a first point of the microfluidic channel and a second point of the microfluidic channel.

19. A method for cell-to-cell DNA, RNA, or other genetic material transfer through use of a water-in-oil emulsion microdroplet-based microfluidic platform for automation and high throughput identification or screening of genetic transfer outcomes, the method comprising:
adding donor cells and target cells to a microfluidic platform thereby forming droplets;
adding an induction media to the microfluidic platform;
adding a signal amplification media to the microfluidic platform;
detecting functionality to allow for analysis of inner content of the droplets; and
sorting the droplets to allow for separation of the droplets based on the analysis of inner content of the droplets.

20. The method of claim 19, wherein the microfluidic device comprises:
a droplet generator comprising an entry point for the donor cells and the target cells;
a first droplet incubation chamber in fluid communication with the droplet generator, wherein the first droplet incubation chamber is configured to allow interaction between the donor cells and the target cells;
an induction media droplet generator comprising an entry point for the induction media;
a first droplet merging region in fluid communication with the first droplet incubation chamber, wherein the induction media droplet generator is configured to release the induction media at a point between the first droplet incubator and the first droplet merging region;
a second droplet incubation chamber in fluid communication with the first droplet merging region, wherein the second droplet incubation chamber is configured to allow for signal induction;
a signal amplification media droplet generator comprising an entry point for the signal amplification media;
a second droplet merging region in fluid communication with the second droplet incubation chamber, wherein the signal amplification media droplet generator is configured to release the signal amplification media at a point between the second droplet incubation chamber and the second droplet merging region;
a third droplet incubation chamber in fluid communication with the second droplet merging region, wherein the third droplet incubation chamber is configured to allow for signal amplification;
a first outlet, wherein the first outlet is configured to release waste cells displaying no DNA transfer; and
a second outlet, wherein the second outlet is configured to recover cells displaying successful DNA transfer.
